(19) United States
(12) Patent Application Publication
(10) Pub. No.: US 2026/0365970 A1
(43) Pub. Date: Nov. 19, 2026

(54) CUSTOMIZABLE 3D CELL CULTURE SYSTEM COMPRISING HYDROGEL-EMBEDDED CELLS AND USES THEREOF

(71) Applicants: Abdellah AJJI, Mont-Royal (CA); Bernard NISOL, Montreal (CA); Derek Hadar ROSENZWEIG, Montreal (CA); Michael Robert WERTHEIMER, Westmount (CA); Mansoureh MOHSENI GARAKANI, Hampstead (CA)

(72) Inventors: Abdellah AJJI, Mont-Royal (CA); Bernard NISOL, Montreal (CA); Derek Hadar ROSENZWEIG, Montreal (CA); Michael Robert WERTHEIMER, Westmount (CA); Mansoureh MOHSENI GARAKANI, Hampstead (CA)

(21) Appl. No.: 19/661,082

(22) Filed: Apr. 28, 2026

Related U.S. Application Data
(62) Division of application No. 17/755,732, filed on Nov. 6, 2020, now abandoned.
(60) Provisional application No. 62/932,759, filed on Nov. 8, 2019.

(51) Int. Cl. C12N 5/00; C12M 1/12; C12N 5/071

(52) U.S. Cl. CPC C12N 5/0062; C12M 25/14; C12N 5/0697; C12N 2503/02; C12N 2533/40

(57) ABSTRACT

A three-dimensional (3D) cell culture system comprising: a solid porous polymeric support, preferably comprising a biocompatible polymer; a first type of cells bound to the solid porous polymeric support; and a biocompatible hydrogel comprising a second type of cells, wherein biocompatible hydrogel is in physical contact with the solid porous polymeric support, is described. Methods for preparing this 3D cell culture system, as well as uses of this system for example for anticancer drug screening, are also described.

### CROSS REFERENCE TO RELATED APPLICATIONS

The present application is a divisional application of U.S. application Ser. No. 17/755,732, filed May 6, 2022, which is a national phase application of PCT application No. PCT/CA2020/051505, filed Nov. 6, 2020, which claims the benefit of U.S. provisional patent application Ser. No. 62/932,759 filed on Nov. 8, 2019. All above applications are incorporated herein by reference in their entirety.

### FIELD OF THE INVENTION

The present disclosure relates to the field of cell culture systems, and more specifically to three-dimensional (3D) cell culture systems that mimic tissues and tumors.

### BACKGROUND OF THE INVENTION

Candidate drugs are typically screened in two-dimensional cultures of cells. Cells cultured in 2D on tissue culture plastic are flat, have 50% of their surface area exposed to tissue culture plastic, and 50% of their cell surface area exposed directly to cell culture media. Under these conditions, the production of extracellular matrix (ECM), which is responsible for signaling between cells and results in tissue specific gene expression, is very limited or absent. As a result, cells cultured in 2D are not phenotypically similar to their in vivo counterparts found in tissues, which comprise both cells and matrix molecules, and have thus significant limitations for drug screening.

Due to the complexity of tumor microenvironments, it is challenging to mimic intercellular interaction in vitro; to do so requires realistic and physiological tissue models. A major issue with conventional two-dimensional (2D) cell cultures is that they cannot reproduce complex in vivo cell-extracellular matrix interactions, nor those between cancer epithelial cells and stromal compartment, which play a crucial role in tumor genesis and progression. Many 3D system culture products have been developed in recent years, including hydrogels, sol gels, ceramic scaffolds, expanded polystyrene supports, permeable membranes, and electrospun nanofiber layers to name a few. Spheroids, an alternate 3D system used in cancer research, are generated only from epithelial tumor cells which lack the heterogeneous cellular components of tumors. Current commercially-available 3D systems such as the Matrigel® products are costly, require multiple steps for implementation, exhibit batch-to-batch variability, have limited mechanical strength and uncontrolled degradation. These products, although providing either a multilayer or etched surface for cell growth, do not truly mimic the more chaotic, 3D fibrous structure of the extracellular matrix deposited by cells growing in living tissue. Because of this, experiments performed on cells growing on material (e.g., rigid polystyrene culture plates) that does not properly represent the geometries and mechanics found in vivo may give results that are not representative of what occurs in living tissue.

There is thus a need for novel (3D) cell culture systems that are customizable and that closely resemble in vivo tissues, tumors and their interfaces.

### SUMMARY OF THE INVENTION

In accordance with the present disclosure, there is provided the following items 1 to 64:
1. A three-dimensional (3D) cell culture system comprising: a first layer comprising a solid porous polymeric support comprising a first type of cells bound thereto; a second layer comprising a biocompatible hydrogel comprising a second type of cells, wherein biocompatible hydrogel is in physical contact with the solid porous polymeric support.
2. The cell culture system of item 1, wherein the solid porous polymeric support comprises a biocompatible polymer.
3. The cell culture system of item 1 or 2, wherein the solid porous polymeric support comprises non-woven nanofibers and/or microfibers.
4. The cell culture system of item 3, wherein the solid porous polymeric support comprises electrospun non-woven nanofibers and/or microfibers.
5. The cell culture system of item 3 or 4, wherein the non-woven nanofibers and/or microfibers have an average length ranging from 10 to 5000 μm.
6. The cell culture system of any one of items 3 to 5, wherein the non-woven nanofibers and/or microfibers have an average diameter ranging from 50 nm to 5 μm.
7. The cell culture system of any one of items 1 to 3, wherein the solid porous polymeric support comprises a 3D-printed polymeric matrix.
8. The cell culture system of any one of items 1 to 7, wherein the biocompatible polymer comprises a poly(lactic acid) (PLA), a poly(lactic-co-glycolic acid) (PLGA), a poly(ε-caprolactone) (PCL), a poly(ethylene terephthalate) (PET), a polyethylene glycol (PEG), a polyurethane (PU), or any combinations thereof.
9. The cell culture system of item 8, wherein the biocompatible polymer comprises a PLA, a PCL, a PU, or any combinations thereof.
10. The cell culture system of item 8 or 9, wherein the PLA comprises poly-L-Lactide (PLLA).
11. The cell culture system of any one of items 1 to 10, wherein the biocompatible hydrogel comprises collagen, fibrin, fibronectin, hyaluronic acid, gelatin, alginate, a gelatinous protein mixture secreted by Engelbreth-Holm-Swarm (EHS) mouse sarcoma cells, de-cellularized patient extracellular matrix, PEG, hydroxyapatite, chitosan, or any combination thereof.
12. The cell culture system of item 11, wherein the biocompatible hydrogel comprises gelatin, alginate or a mixture thereof.
13. The cell culture system of item 12, wherein the biocompatible hydrogel comprises a mixture of gelatin and alginate.
14. The cell culture system of any one of items 1 to 13, wherein the first type of cells comprises epithelial cells, endothelial cells, osteoblasts, stromal cells, immune cells, adipocytes, chondrocytes, stem cells, neurons, glial cells, astrocytes, or any combination thereof.
15. The cell culture system of item 14, wherein the first type of cells comprises epithelial cells, endothelial cells, osteoblasts, stromal cells, or any combination thereof.
16. The cell culture system of item 14 or 15, wherein the stromal cells are fibroblasts.
17. The cell culture system of any one of items 1 to 16, wherein the second type of cells comprises tumor cells.
18. The cell culture system of item 17, wherein the second type of cells further comprises tumor stem-like cells, tumor-associated cells, endothelial cells, immune cells, endothelial cells, fibroblasts, epithelial cells, stem cells, or any combination thereof.
19. The cell culture system of any one of items 1 to 18, wherein the biocompatible hydrogel is superposed on the top of the solid porous polymeric support.
20. The cell culture system of any one of items 1 to 19, further comprising a third layer, or a third layer and a fourth layer.
21. The cell culture system of item 20, wherein the third layer comprises a solid porous polymeric support comprising a third type of cells bound thereto.
22. The cell culture system of item 20 or 21, wherein the second layer is between the first layer and the third layer.
23. A method for preparing a three-dimensional (3D) cell culture system, the method comprising: (i) providing a functionalized solid porous polymeric support; (ii) seeding a first type of cells on the functionalized solid porous polymeric support to attach the first cell type on the solid porous polymeric support; (iii) contacting the solid porous polymeric support of step (ii) with a biocompatible hydrogel comprising a second type of cells, thereby obtaining the 3D culture system.
24. The method of item 23, wherein the solid porous polymeric support comprises a biocompatible polymer
25. The method of item 23 or 24, wherein the solid porous polymeric support comprises non-woven nanofibers and/or microfibers.
26. The method of item 25, wherein the solid porous polymeric support comprises electrospun non-woven nanofibers and/or microfibers.
27. The method of item 25 or 26, wherein the non-woven nanofibers and/or microfibers have an average length ranging from 10 to 5000 μm.
28. The method of any one of items 25 to 27, wherein the non-woven nanofibers and/or microfibers have an average diameter ranging from 50 nm to 5 μm.
29. The method of any one of items 23 to 25, wherein the solid porous polymeric support comprises a 3D-printed polymeric matrix.
30. The method of any one of items 23 to 29, wherein the biocompatible polymer comprises a poly(lactic acid) (PLA), a poly(lactic-co-glycolic acid) (PLGA), a poly(ε-caprolactone) (PCL), a poly(ethylene terephthalate) (PET), a polyethylene glycol (PEG), a polyurethane (PU), or any combinations thereof.
31. The method of item 30, wherein the biocompatible polymer comprises a PLA, a PCL, a PU, or any combinations thereof.
32. The method of item 30 or 31, wherein the PLA comprises poly-L-Lactide (PLLA).
33. The method of any one of items 23 to 32, wherein the biocompatible hydrogel comprises collagen, fibrin, fibronectin, hyaluronic acid, gelatin, alginate, a gelatinous protein mixture secreted by Engelbreth-Holm-Swarm (EHS) mouse sarcoma cells, de-cellularized patient extracellular matrix, PEG, hydroxyapatite, chitosan, or any combination thereof.
34. The method of item 33, wherein the biocompatible hydrogel comprises gelatin, alginate or a mixture thereof.
35. The method of item 34, wherein the biocompatible hydrogel comprises a mixture of gelatin and alginate.
36. The method of any one of items 23 to 35, wherein the first type of cells comprises epithelial cells, endothelial cells, osteoblasts, stromal cells, immune cells, adipocytes, chondrocytes, stem cells, neurons, glial cells, astrocytes, or any combination thereof.
37. The method of item 36, wherein the first type of cells comprises epithelial cells, endothelial cells, osteoblasts, stromal cells, or any combination thereof.
38. The method of item 36 or 37, wherein the stromal cells are fibroblasts.
39. The method of any one of items 23 to 38, wherein the second type of cells comprises tumor cells.
40. The method of item 39, wherein the second type of cells further comprises tumor stem-like cells, tumor-associated cells, endothelial cells, immune cells, endothelial cells, fibroblasts, epithelial cells, stem cells, or any combination thereof.
41. The method of any one of items 23 to 40, wherein the biocompatible hydrogel is superposed on the top of the solid porous polymeric support.
42. The method of any one of items 23 to 41, wherein the method further comprises, prior to step (i), submitting the solid porous polymeric support to plasma treatment to obtain the functionalized solid porous polymeric support.
43. The method of item 42, wherein the plasma treatment is performed by plasma-enhanced chemical vapor deposition (PECVD).
44. The method of item 42 or 43, wherein the plasma is an 02 plasma, an NH3 plasma, or an oxygen-, sulfur- or nitrogen-rich plasma-polymer.
45. The method of item 44, wherein the oxygen- or nitrogen-rich plasma-polymer is PP-[oxygen-rich ethylene] (PPE:O) or PP-[nitrogen-rich ethylene] (PPE:N).
46. The method of item 44, wherein the oxygen- or nitrogen-rich plasma polymer is produced using a hydrocarbon source gas comprising butadiene, acetylene, propylene, or butylene.
47. The method of item 44, wherein the oxygen- or nitrogen-rich plasma polymer is produced using a volatile organic source gas or vapor that contains a desired oxygen- or nitrogen functionality or functionalities
48. The method of item 47, wherein the volatile organic source gas or vapor comprises an organic acid, an alcohol, an ester or an amino-compound.
49. The method of item 48, wherein the organic acid is acrylic acid.
50. The method of item 48, wherein the ester is ethyl lactate (EL).
51. The method of item 48, wherein the amino-compound is allylamine (AAm).
52. The method of any one of items 23 to 51, further comprising culturing the 3D culture system.
53. The method of item 52, wherein at least a portion of the second type of cells migrate at the surface and/or into the solid porous polymeric support during said culturing.
54. A cell culture device comprising the cell culture system of any one of items 1 to 22.
55. The cell culture device of item 54, which is a petri dish or a multi-well plate.
56. Use of the cell culture system of any one of items 1 to 22 for assessing the effect of an agent on the first and/or second types of cells defined in any one of items 1 to 22.
57. The use of item 56, wherein the effect comprises change in gene and/or protein expression, cell death, cell differentiation, cell proliferation and/or cell migration.
58. The use of item 56 or 57, wherein the agent is a candidate anti-tumor agent.
59. A method for assessing the effect of an agent on the first and/or second types of cells defined in any one of items 1 to 22, the method comprising contacting the cell culture system of any one of items 1 to 22 with said agent.
60. The method of item 59, wherein the effect comprises change in gene and/or protein expression, cell death, cell differentiation, cell proliferation and/or cell migration.
61. The method of item 59 or 60, wherein the agent is a candidate anti-tumor agent.
62. A method for determining whether a test agent inhibits the growth and/or migration of cells of interest comprising contacting the cell culture system of any one of items 1 to 22 in presence or absence of the test agent, wherein the cells of interest are the second type of cells defined in any one of items 1 to 22; and determining the number of the cells of interest in the cell culture system, wherein a lower number of the cells of interest in the presence of the test agent relative to the absence thereof is indicative that the test agent inhibits the growth and/or migration of the cells of interest.
63. The method of item 62, wherein the cells of interest are tumor cells, and wherein the test agent is a candidate anti-tumor agent.
64. The method of item 62 or 63, wherein the method comprises determining the number of the cells of interest in the second layer.

### BRIEF DESCRIPTION OF THE DRAWINGS

In the appended drawings:

FIG. 1 is a schematic representation of a representative process to prepare a 3D cell culture system according to an embodiment of the present disclosure: (i) electrospinning of polymeric scaffold; (ii) bio-activation or functionalization by plasma treatment/coating; (iii) seeding of a first type of cells; (iv) deposit of hydrogel with second type of cells (e.g., tumor cells) and migration of the latter upon culture.

FIGS. 2A and 2B show the results of cell-seeding experiments (step (iii) in FIG. 1 above) using breast cancer cells (FIG. 2A) or osteoblasts on (FIG. 2B) poly-lactic acid (PLA) electrospun mats of varying fiber diameters (“small”, “medium”, and “large”) functionalized by NH₃, O₂ or L-PPE:N plasma treatment. The ordinate label “percent” refers to the percentage of cells that were found adhering within the mats 24 hours after seeding.

FIG. 3 shows confocal fluorescent images of the surface of scaffolds functionalized by O₂ or L-PPE:N plasma treatment before (day 0, left panels) and after scraping hydrogel-containing tumor cells after 21 days of cells culture (right panels), in order to evaluate tumor cell migration from the hydrogel into the scaffolds.

FIG. 4A shows the number of tumor cells at the surface of PLA electrospun mats functionalized by O₂, NH₃, or L-PPE:N or L-PPE:O plasma treatment.

FIG. 4B shows the number of tumor cells in the depth of PLA electrospun mats functionalized by O₂, NH₃, or L-PPE:N or L-PPE:O plasma treatment.

FIGS. 5A-C show fiber diameter distribution in PLA (FIG. 5A), poly-caprolactone (PCL) (FIG. 5B) and polyurethane (PU) (FIG. 5C) electrospun mats.

FIG. 6 depicts confocal fluorescent images showing tumor migration on PLA (upper panels), PCL (middle panels) and PU (lower panels), O₂ plasma-treated nanofibrous scaffolds at day 1 (left panels), day 3 (middle panels) and day 7 (right panels) (magnification 4×).

FIG. 7A depicts confocal fluorescent images showing tumor migration on PLA (upper panels), PCL (middle panels) and PU (lower panels), at day 7 in nanofibrous scaffolds treated (left panels) or not (Ctrl, right panels) with O₂ plasma (magnification 4×).

FIG. 7B is a graph showing the number of MDA-MB 231 breast cancer cells that migrated from the hydrogel to the PLA scaffolds after 7 days. PLA electrospun mats (medium size) treated with three different plasma coatings including L-PPE:O (ethylene/argon+oxygen mixture gas in low pressure); plasma polymers derived from monomers, ethyl lactate (EL), and allylamine in atmospheric pressure discharges. The PLA scaffolds were seeded with 20,000 fibroblasts first and then a hydrogel droplet (Alginate/Gelatin) containing 20,000 MDA-MB 231 breast cancer cells was placed on top and tumor migration was monitored over 7 days after cell culture.

FIG. 8A is a graph showing the number of human dental pulp stem cells initially adhered to PLA, PCL and PU microporous 3D-printed scaffolds treated (left bars) or not (Ctrl, right bars) with O₂ plasma.

FIG. 8B is a graph showing the number of human dental pulp stem cells initially adhered to PLA microporous 3D-printed scaffolds treated with L-PPE:N, O₂ or NH₃ plasma, relative to untreated control.

FIGS. 9A-D depict images of human dental stem cell growth, propagation and network formation into the pores of L-PPE:N- (FIG. 9A), NH₃- (FIG. 9B) or O₂- (FIG. 9C) plasma-treated and non-treated (FIG. 9D) PLA 3D printed scaffold after 21 days of culture.

FIG. 10 is a graph showing the measurement of the network area produced by dental stem cells inside the pores of PLA 3D-printed scaffold functionalized by L-PPE:N, O₂ or NH₃ plasma treatment, or not functionalized (Ctrl), after 21 days of culture.

FIG. 11A depicts confocal fluorescent images of tumor migration monitoring at day 7 after addition of different concentrations (0, 0.05, 0.1, 0.5, 1 or 2 μM) of Doxorubicin (Drug) to PLA nanofibrous mats cultured with breast cancer cells.

FIG. 11B is a graph showing the measurement of the number of tumor cells migrated to the surface of PLA mats at different concentrations (0, 0.05, 0.1, 0.5, 1 or 2 μM) of Doxorubicin. Experiments were performed in triplicate and the error bars in the graph show the standard deviation.

FIG. 12A depicts confocal fluorescent images of the migration of patient-derived tumor cells (BMP4, left image) or cell line tumor cells (MDA-MB 231, right panel) on PP-EL plasma-treated PLA mats after 7 days.

FIG. 12B depicts confocal fluorescent images of tumor migration monitoring at day 7 after addition of different concentrations (0, 0.05, 0.1, or 0.5 μM) of Doxorubicin (Drug) to PP-EL plasma-treated PLA nanofibrous mats cultured with BMP4 tumor cells.

FIG. 12C is a graph showing the measurement of the number of BMP4 tumor cells migrated to the surface of PP-EL plasma-treated PLA mats at different concentrations (0, 0.05, 0.1, or 0.5 μM) of Doxorubicin. Experiments were performed in triplicate and the error bars in the graph show the standard deviation.

FIG. 13 is a graph showing the comparison of a 3D cell culture system according to an embodiment of the present disclosure (left bars) and a Matrigel® system (right bars) in a tumor metastasis evaluation test. The number of migrated tumor cells was measured at different concentrations (0, 0.05, 0.1 and 0.5 μM) of Doxorubicin. Experiments were performed in triplicate and the error bars in the graph show the standard deviation.

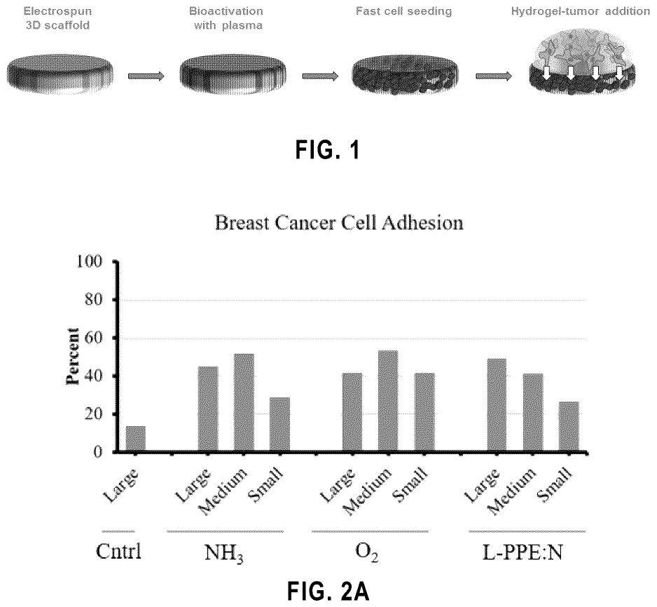

### DETAILED DESCRIPTION OF THE INVENTION

The present inventors have shown that plasma-treated or -coated 3D polymer scaffolds (“PP-3DS”), either obtained by electrospinning or 3D printing, supplemented by cell-seeded hydrogel for cell transfer to the PP-3DS, constitute a relevant and flexible microenvironment for growing tissue models that mimic physiological conditions of various types of tissues. The 3D cell culture system obtained was shown to possess the appropriately controllable degrees of mechanical rigidity, porosity and biochemical capabilities of living tissues, and enable in-depth study of cancerous tissues by known bioengineering and biological methods, for example in high-throughput screening of anticancer drugs.

The present disclosure provides a three-dimensional (3D) cell culture system comprising:
a first layer comprising a functionalized solid porous polymeric support, preferably comprising a biocompatible polymer, and a first type of cells bound to the solid porous polymeric support;
a second layer comprising a biocompatible hydrogel comprising a second type of cells, wherein biocompatible hydrogel is in physical contact with the solid porous polymeric support.

The present disclosure provides a kit for preparing a three-dimensional (3D) cell culture system, the kit comprising: a functionalized solid porous polymeric support preferably comprising a biocompatible polymer; and a biocompatible hydrogel. In an embodiment, the functionalized solid porous polymeric support comprises a first type of cells bound thereto. In an embodiment, the biocompatible hydrogel comprises a second type of cells. In addition to these components, the kit may also comprise suitable containers, such as flasks, vials or multi-well plates to hold its components, preferably separately for each component or medium.

### Solid Matrix Support

The functionalized solid porous matrix support, preferably a polymeric support, is made of a preferably biocompatible material (such as a polymer), and wherein the surface of the matrix support is enriched in functional groups (e.g., O-containing and/or N-containing functional groups) that increase surface hydrophilicity and facilitate the binding of biomolecules such as proteins (e.g., integrin receptors) that are present at the surface of the cells, thereby improving cell adhesion/attachment to the support.

In embodiments, the solid porous matrix support is a polymeric support, i.e. is made of a polymer or mixture of polymers. In embodiments, the solid porous polymeric support is a non-woven nanofiber and/or microfiber mat, for example a mat of electrospun nanofibers or microfibers, or a 3D-printed matrix.

The term “biocompatible” as used herein means that the material is not cytotoxic at the concentration used in the system.

Non-woven nanofiber or microfiber mat refers to a mat of individual fibers or filaments which are interlaid and positioned in a random (or a partially-aligned) manner to form a planar material substantially without identifiable pattern, as opposed to a knitted or woven fabric. Non-woven nanofiber or microfiber mats may be prepared by methods well known in the art, such as electrospinning, melt spinning (melt-blowing), dry spinning, wet spinning or extrusion. In an embodiment, the non-woven nanofiber or microfiber mat is an electrospun mat.

In more specific embodiments, the solid porous polymeric support is a non-woven nanofiber and/or microfiber mat, preferably a mat of electrospun nanofibers and/or microfibers. In such embodiments, the diameter of the nanofibers/microfibers can vary, for example from 100 nm to a few microns, for example from 100 nm to 5 μm, from 100 nm to 2 μm, from 100 nm to 1.5 μm, or from 100 nm to 1 μm. In another embodiment, the diameter of the nanofibers/microfibers is from 100 to 300 nm. In another embodiment, the diameter of the nanofibers/microfibers is from 500 to 700 nm). In another embodiment, the diameter of the nanofibers/microfibers is from 1.0 to 1.5 μm). Fiber orientation can also vary. The mean diameter of the fibres may be measured, for example, by Scanning Electron Microscopy (SEM).

The solid porous polymeric support may comprise only nanofibers, only microfibers, or a mixture of nanofibers and microfibers. The mixture of nanofibers and microfibers may comprise any suitable proportion of nanofibers and microfibers, for example, 5-95 wt % of microfibers and 5-95 wt % nanofibers, or 10-90 wt % of microfibers and 10-90 wt % nanofibers, or 20-80 wt % of microfibers and 20-80 wt % nanofibers, or 30-70 wt % of microfibers and 30-70 wt % nanofibers, or 40-60 wt % of microfibers and 40-60 wt % nanofibers, or 50 wt % of microfibers and 50 wt % nanofibers, or 50-90 wt % of microfibers and 10-50 wt % nanofibers, or about 90 wt % of microfibers and about 10 wt % nanofibers, or about 80 wt. % of microfibers and about 20 wt % nanofibers, or about 70 wt % of microfibers and about 30 wt % nanofibers, or about 60 wt % of microfibers and about 40 wt % nanofibers.

In alternative embodiments, the solid porous support is a 3D-printed matrix, preferably a 3D-printed polymeric matrix.

The solid porous support can be of any shape and size. Preferably, it is up to about 2000 μm, preferably about 1000 μm thick, more preferably about 500 μm and most preferably about 250 μm in thickness. In embodiments, the polymeric support has a thickness of about 10 μm to about 2000 μm, about 50 μm to about 1500 μm, about 100 μm to about 1000 μm, about 100 μm to about 500 μm, about 100 to about 200 μm, about 150 to about 250 μm, about 200 μm to about 300 μm, or about 200-250 μm.

As noted above, the support is porous. This porosity is an interconnected porosity, meaning that the pores are generally connected to each other allowing fluid (gas, liquid) and even cell passage in the support.

The skilled person would understand that the porosity, pore size, and nature of the biocompatible material (e.g., polymer) can be adjusted depending of the type of tissue to be mimicked and/or the type of cells to be used. Indeed, all these factors will affect the mechanical properties of the support, which can thus be selectively adjusted as desired. Typically, use of a stiffer biocompatible material (e.g., inorganic material, polymer) will yield a stiffer support, which may be useful to mimic hard tissues such as bones or cartilages. However, for a given biocompatible polymer, increasing porosity and/or pore size may yield a more flexible material.

In embodiments, the matrix support has a porosity of at least about 30, 40 or 50%. In embodiments, the matrix support has a porosity of 95% or 90% or less. In preferred embodiments, the matrix support has a porosity between about 40% and about 90%, between about 50% and about 90%, between about 60% and about 90%, or between about 70% and about 90%.

In embodiments, the matrix support has a mean pore size of at least 50, 100, 150, 200, 250 or 300 nm. In embodiments, the matrix support has a mean pore size of about 10 μm or less. In preferred embodiments, the matrix support has a mean pore size of about 100 or 200 nm to about 10, 9, 8, 7 or 6 μm, about 200 to about 6 μm, about 300 to about 6 μm, or about 300 to about 5 μm.

The mean pore diameter may be estimated theoretically with a simplification of the model of Eichhorn and Sampson [S. J. Eichhorn, W. W. Sampson, Statistical geometry of pores and statistics of porous nanofibrous assemblies, J R Soc Interface 2(4) (2005) 309-318], in which it is related to the fiber diameter d and the total porosity E of the scaffold as indicated in the equation below [S. Soliman, S. Sant, J. W. Nichol, M. Khabiry, E. Traversa, A. Khademhosseini, Controlling the porosity of fibrous scaffolds by modulating the fiber diameter and packing density, J. Biomed. Mater. Res. A 96(3) (2011) 566-74].

$$r = - \frac{d}{ln ε}$$

Porosity may be determined by density measurements using methods known in the art, such as quantitative micro-computed tomographic (micro-CT) analysis.

In an embodiment, the biocompatible matrix comprises or is a biocompatible polymer. The biocompatible polymer can comprise any biocompatible polymer or combinations thereof known for use as scaffold for cell culture, for example polyolefin, a polystyrene, a cellulose, a cellulose acetate, a cellulose derivative, a poly(lactic acid) (PLA), a polylactic-co-glycolic acid (PLGA), a poly(methyl methacrylate), a polyacrylonitrile, a polyvinylidene difluoride, a poly(vinyl chloride]), a poly(vinyl acetate), a poly(ethylene oxide), a polycaprolactam, a polyacetal, a polycaprolactone (PCL), a polyetherimide, a polyethylene glycol (PEG), a polyamide, a polyurea, a polyester, a polycarbonate, a polyurethane, a polyimide, a polysiloxane, or a polysulfone, or any combination thereof. The biocompatible matrix may comprise a blend of two or more polymers, a copolymer (which may for instance be a block copolymer), or a blend of a polymer with an inorganic material.

In embodiments, the biocompatible polymer comprises or is:
a poly(lactic acid), including poly(L-lactic acid) (PLLA), poly(D-lactic acid) (PDLA), and poly(DL-lactic acid) comprising any ratio of D- and L-lactic acid repeat unit. The poly(lactic acid) may have a weight average molecular weight (Mw) of about 50,000 g/mol to 400,000 g/mol, e.g., a Mw of about 180,000 g/mol to 260,000 g/mol;
poly(lactic-co-glycolic acid) (PLGA), including poly(L-lactic-co-glycolic acid), poly(D-lactic-co-glycolic acid), and poly(DL-lactic-co-glycolic acid) comprising any ratio of D- and L-lactic acid repeat unit;
poly(ε-caprolactone) (PCL), preferably a PCL having an average Mₙ of between about 40,000 to about 120,000, or between about 60,000 to 100,000, or between 70,000 and 90,000, more preferably about 80,000.
poly(ethylene terephthalate) (PET),
polyethylene glycol (PEG), or
a polyurethane (PU), including poly[4,4′-methylenebis(phenyl isocyanate)-alt-1,4-butanediol/di(propylene glycol)/polycaprolactone].

In preferred embodiments, the biocompatible polymer comprises or is:
a poly(lactic acid), most preferably a poly(lactic acid) having one or more of the following properties:

| | | |
|---|---|---|
| Properties | Value | ASTM Method |

Physical properties

| | | |
|---|---|---|
| Specific Gravity | 1.24 | D792 |
| MFR, g/10 min (210 °C, 2.16 kg) | 7 | D1238 |
| Melt density (g/cc) | 1.08 at 230 °C | |

Mechanical properties

| Tensile Strength @ Break, psi (MPa) | 7,700 | (53) | D882 |
|---|---|---|---|
| Tensile Yield Strength, psi (MPa) | 8,700 | (60) | D882 |
| Tensile Modulus, kpsi (GPa) | 500 | (3.5) | D882 |
| Tensile Elongation, % | 6.0 | | D882 |
| Notched Izod Impact, ft-lb/in (J/m) | 0.3 | (16) | D256 |

| | |
|---|---|
| Melting Point (°C) | 155-170 |

for example Ingeo™ Biopolymer 4032D from NatureWorks.
poly(ε-caprolactone), such as PCL having an average Mₙ of between about 60,000 to 100,000, or between 70,000 and 90,000, more preferably about 80,000, such as the PCL commercialized by Millipore Sigma under Cat No. 440744;
a polyurethane, such as poly[4,4′-methylenebis(phenyl isocyanate)-alt-1,4-butanediol/di(propylene glycol)/polycaprolactone] (CAS Number 68084-39-9).

In another embodiment, the biocompatible matrix comprises an inorganic solid material, such as minerals and ceramics (e.g., silica, alumina, hydroxyapatite), which may be useful to mimic the properties of certain tissues, such as bones or teeth.

As noted above, the surface of the matrix support is enriched in functional groups, such as sulfur (S)-containing, phosphorous (P)-containing, oxygen (O)-containing and/or nitrogen (N)-containing functional groups. This means that the surface of the matrix support has been treated in some way to increase the number of functional groups, preferably O-containing and/or N-containing functional groups, chemically-bound (attached) to the surface compared to the untreated surface of the matrix support (i.e. the bare biocompatible matrix). In an embodiment, the functional groups are O-containing and/or N-containing functional groups. Some preferred O- or N-containing groups may be hydroxyl (—OH), carboxylic acid (—COOH) or primary amine (C—NH₂), but these are only some examples. Persons skilled in the art will recognize that many other such groups will be able to fulfill this role.

Of note, the internal and the external surface of the matrix support is enriched in functional groups (e.g., O-containing and/or N-containing functional groups). This means that the walls of the pores in the matrix support also bear such groups. As described below, preferred surface treatments include plasma (both “cold” low-pressure plasma and its atmospheric-pressure counterpart), which allow in-depth surface modifications, because the plasmas' active precursor particles can readily penetrate into and travel through the pores of the porous 3D matrix support. It is noteworthy that only a very shallow surface-near region of the solid needs to be affected by the plasma treatment, because biomolecules and/or cells “see” only the first nanometer(s).

Non-limiting examples of O-containing functional groups include —COOH, —OH, —CO, —C═O, for example.

Non-limiting examples of N-containing functional groups include —NH₂, ═NH, —NO, for example.

In embodiments, the surface of the polymeric support bears:
(a) grafted O-containing, N-containing and/or S-containing functional groups, or
(b) a coating comprising O-containing, N-containing and/or S-containing functional groups.
O-containing, N-containing and S-containing functional groups can be individually grafted onto the surface of the polymeric support by exposing the polymeric support to a plasma of a non-polymerizing nitrogen-, oxygen- or sulfur-containing gas. Thus, in embodiments, the surface of the polymeric support is a plasma-treated surface. In preferred embodiments, the surface of the polymeric support is a low-pressure plasma-treated surface. The term “plasma”, also known as the “fourth state of matter”, thereby refers to an electrically conducting (but electrostatically neutral) process gas phase involving free electrons and ions (in approximately equal number densities), and energetic photons. Plasma is commonly generated by means of suitable electric field generating means, such as electrodes, in a vacuum chamber (using radio- or microwave frequency “RF or MW plasma”), but it can also be generated using capacitive or inductive methods, or microwave radiation. Suitable “cold” plasma (gas near ambient temperature, ca. 300 K) can also be obtained at atmospheric pressure, ca. 100 kPa, as is well-known to those skilled in the art. The most important process gases are oxygen, hydrogen, nitrogen, argon, helium, air, water vapor, hydrocarbons, organic compound gases and vapors and mixtures thereof, but other process gases may be used as well-known to persons skilled in applied plasma science.

Of note, a “non-polymerizing” nitrogen- or oxygen-containing gas is a gas (containing N and/or O) that will not polymerize on the surface during plasma treatment. Typically, this means that the gas does not comprise carbon or silicon atoms, which tend to “polymerize” or lead to thin film deposits in such conditions. Of further note, “polymerizing” gases are useful, because they will yield a coating (as in (b) above) rather than simply new functional groups (as in (a) above).

Non-limiting examples of non-polymerizing nitrogen- or oxygen-containing gases for plasma treatment include N₂ or NH₃ (for grafting N-containing functional groups such as amines, imine, etc.); O₂, CO₂ or H₂O (for grafting O-containing functional groups such as —OH, —CO—, and —COOH); air, O₂+N₂ mixtures, NOx compounds, etc. (for grafting oxygen and nitrogen-containing groups, such as amides), H₂S and/or CS₂ (for grafting sulfur-containing groups, such as thiols), but these are only a few examples among many others known to persons skilled in applied plasma science.

Chemical bonding of O-containing, N-containing and S- or P-containing functional groups may be performed by addition of ultra-thin plasma polymer coatings to the polymer surface, that can be obtained by:
(I) mixing “non-polymerizing” gases noted above with a hydrocarbon “monomer”. Non-limiting examples of suitable hydrocarbon “monomers”: hydrocarbon source gas, preferably unsaturated (e.g., ethylene, butadiene, acetylene, propylene, butylene, etc.); these can yield plasma-deposited (plasma-polymerized, PP) coatings comprising O-containing, N-containing and/or S-containing functional groups such as:
PP-[oxygen-rich ethylene] (PPE:O); PP-[oxygen-rich butadiene] (PPB:O), etc. by:
   Cold plasma at low pressure (e.g., L-PPE:O);
   Cold plasma at “high” atmospheric pressure (e.g., H-PPE:O)
PP-[nitrogen-rich ethylene] (PPE:N):
   Cold plasma at low pressure (e.g., L-PPE:N);
   Cold plasma at atmospheric pressure (e.g., H-PPE:N);
PP-[nitrogen- and oxygen rich ethylene] (PPE:N,O):
   Cold plasma at low pressure (e.g., L-PPE:N,O);
   Cold plasma at atmospheric pressure (e.g., H-PPE:N,O);
PP-[sulfur-rich ethylene] (PPE:S):
   Cold plasma at low pressure (e.g., L-PPE:S); Cold plasma at atmospheric pressure (e.g., H-PPE:S).
(II) by using a “monomer” that satisfies these requirements:
   (i) organic precursor compounds that already contain the desired above-noted functional groups (e.g., amines, imine, —OH, —CO—, —COOH, amides and/or thiols);
   (ii) that are highly volatile, i.e. are either gaseous at 300 K, or have sufficiently high vapor pressure. This includes oxygen-rich, nitrogen-rich and sulfur-rich plasma polymer produced with volatile organic source gas or vapor that contains (or from which plasma activation will result in) a desired oxygen-, nitrogen- or sulfur-functionality or functionalities (the “monomer”)
oxygen-rich:
   acids (e.g., acrylic, acetic, formic, etc.)
   alcohols (e.g., ethanol, propanol, ethane-1,2-diol, allyl alcohol, hydroxyethyl methacrylate, etc)
   esters (e.g., ethyl lactate, propyl isobutyrate, allyl methacrylate, etc)
   anhydrides (e.g., acetic anhydride, propionic anhydride, isobutyric anhydride, methacrylic anhydride, etc.)
nitrogen-rich:
   amino-compounds such as allylamine, propylamine, propargylamine, ethylene diamine, n-heptylamine, cyclopropylamine, diaminocyclohexane, butylamine, etc.
sulfur-rich:
   organic molecules from the family of thiols, such as methyl mercaptan, ethyl mercaptan, n-propyl mercaptan, 2-propanethiol, allyl mercaptan, tert-Butyl mercaptan, etc.

All of the above can be achieved at both low pressure and at atmospheric pressure, the latter for example in a cold dielectric barrier discharge (DBD) plasma, or in a suitable plasma jet, by mixing the reagent gas (or gas mixture) with a suitable inert carrier gas such as argon or helium.

In an embodiment, the matrix support is functionalized or coated by PP ethyl-lactate (PP-EL), PP-allylamine (PP-AAm), PP-[nitrogen-rich plasma-polymerized ethylene] (PPE:N), such as (L-PPE:N) or (H-PPE:N), PP-[oxygen-rich plasma-polymerized ethylene] (PPE:O), such as (L-PPE:O) or (H-PPE:O).

Other low- or atmospheric-pressure plasma polymerized (L-PP or H-PP) coatings containing O— or/and N— may be obtained using hydrocarbon precursor gases or vapors such as butadiene, acetylene, propylene, etc., as is well known to persons skilled in plasma polymerization, wherein “PP” stands for “plasma polymerized” and “L-PP” stands for “low-pressure plasma polymerized”, and “H-PP” stands for “high-pressure plasma polymerized”. Indeed, all these coatings can be produced by plasma-enhanced chemical vapor deposition (PECVD) using various polymerizing nitrogen- or oxygen-containing gases or gas/vapor mixtures.

PP ethyl-lactate (PP-EL) can be produced by PECVD using ethyl-lactate vapor, as described in Nisol et al., incorporated herein by reference.

PP allylamine (PP-AAm) can be produced by PECVD using allylamine vapor, as described in Wyrwa et al., incorporated herein by reference.

A preferred coating is PP-[nitrogen-rich ethylene] (PPE:N), such as (L-PPE:N) and (H-PPE:N), which is an amine-rich plasma-polymerized ethylene coating, prepared by plasma-enhanced chemical vapor deposition (PECVD) using ethylene and ammonia, as described by Savoji et al. (2014), “Electrospun Nanofiber Scaffolds and Plasma Polymerization: A Promising Combination Towards Complete, Stable Endothelial Lining for Vascular Grafts”, Macromol. Biosci., 14, 1084-1095, incorporated herein by reference.

The plasma polymer coatings produced by PECVD are thin, typically up to about 1 micrometer thick, but preferably only some tens of nanometers. In preferred embodiments, the coating is about 10-500 nm, about 20-200 nm, about 50-150 nm, or about 100 nm thick.

In preferred embodiments, the surface of the polymeric support bears:
grafted O-containing functional groups obtained by exposing the polymeric support to a O₂, H₂O or CO₂ plasma, for example, (preferably low-pressure plasma), or
grafted N-containing functional groups obtained by exposing the polymeric support to a N₂ or NH₃ plasma (preferably low-pressure plasma),
a PP-[nitrogen- or oxygen-rich ethylene] (PPE:N or PPE:O), preferably (L-PPE:N or L-PPE:O) coating.

In most preferred embodiments, the surface of the polymeric support bears a PP-[nitrogen-rich ethylene](PPE:N), preferably (L-PPE:N) coating, or a PP-[oxygen-rich ethylene] (PPE:O), preferably (L-PPE:O) coating. However, as known to skilled persons, many other plasma-polymers may also be suitable.

Herein, as well-known to the skilled person, “low-pressure plasma” is a plasma produced at a pressure lower than atmospheric pressure. Typical operating pressure for low-pressure plasma range from about 10 milliTorr (1.33 Pa) to a few torr (several hundred Pa). Low-pressure plasma coatings can be prepared using, e.g., partial vacuum of typically ca. 100 milliTorr (13.3 Pa), in radio-frequency (r.f., 13.56 MHz) capacitively-coupled discharge plasmas.

Plasma treatments such as low-pressure and high-pressure (atmospheric)-pressure (760 Torr or 100 kPa) plasma treatments have the advantage of allowing deep penetration of the (plasma) active species into and through interconnected pores of the matrix support, probably as deep as 1000 μm or more, thanks to their large mean-free-path lengths and/or other physical reasons, for example energetic ultraviolet photons, and others. The beneficial result, of course, is nearly-uniform surface-chemical composition, hence nearly-uniform cell response.

The first type of cells bound to the polymeric support may be any type of cells (or combination of cells) suitable to mimic a tissue or organ of interest. Thus, the type(s) of cells is selected according to the tissue or organ that the cell culture system intends to mimic. The first type of cells may be, e.g., connective tissue cells (e.g., stromal cells, fibroblasts), endothelial cells, epithelial cells, neuroglial cells, neurones, muscle cells (e.g., skeletal, cardiac, or smooth muscle cells), cartilage cells (e.g., chondrocytes), bone cells (e.g., osteoblasts, osteoclasts, osteocytes, lining cells), skin cells (e.g., keratinocytes, melanocytes, Langerhans cells), immune cells (e.g., lymphocytes, macrophages/monocytes, neutrophils, etc.), astrocytes or any combination thereof, the list of course not being exhaustive. The first type of cells may be primary cells or a cell line, malignant or non-malignant (normal) cells. In an embodiment, the first type of cells are not tumor or malignant cells. In another embodiment, the first type of cells are progenitor, such as stem cells, to any tissue, such as totipotent stem cell, pluripotent stem cell, multipotent stem cell, mesenchymal stem cell, neural stem cell, hematopoietic stem cell, pancreatic stem cell, dental pulp stem cell (which may differentiate toward bone, cartilage, fat, or muscle lineage), cardiac stem cell, embryonic stem cell, embryonic germ cell, neural stem cell, especially a neural crest stem cell, kidney stem cell, hepatic stem cell, lung stem cell, hemangioblast cell, induced pluripotent stem cells (IPSC), and endothelial progenitor cell. Such progenitor cells may be induced to differentiate into a cell type of interest under appropriate culture conditions, e.g., by contacting the progenitor cells with tissue-specific growth or differentiation factor(s). The cell may be a primary cell, a cell line, a genetically-engineered cell, etc.

The amount of the first type of cells incorporated in the matrix support is selected on the basis of various factors, including the tissue or organ that the cell culture system intends to mimic and the size of the matrix support. The number of cells may be, e.g., at least 10² or 10³ cells and up to 10⁸ or 10⁹ cells, from 10³ to 10⁷, from 10³ to 10⁶, or from 10⁴ to 10⁵ cells. In an embodiment, the cell density in the matrix support is from about 10² to about 10⁷ cells/cm³, about 10³ to about 10⁷ cells/cm³, about 10⁴ to about 10⁶ cells/cm³, or about 10⁵ to about 10⁶ cells/cm³.

In addition to the components defined above, the first layer may further comprise other materials such as extracellular matrix molecules, proteins, peptides, nucleic acids, dyes (fluorescent dyes), etc.

### Hydrogel

The 3D cell culture system also comprises a biocompatible hydrogel comprising a second type of cells, for example migrating cells of interest (cancer cells or others). The biocompatible hydrogel is put in contact with, e.g. layered on top of, the matrix support to allow the growth, differentiation migration of the second type of cells at the surface and/or into the matrix support.

As well-known to the skilled person, a hydrogel is a three-dimensional (3D) network of a hydrophilic polymer that can swell in water and hold a large amount of water while maintaining its structure due to chemical or physical cross-linking of individual polymer chains.

Thus, the hydrogel comprises water and a hydrophilic polymer. The hydrophilic polymer is present in a concentration such that a gel is obtained, which concentration will depend on the exact nature of the polymer used as well as the desired mechanical properties of the hydrogel (e.g., strength, viscosity).

Non-limiting examples of hydrophilic polymers include collagen, fibrin, fibronectin, hyaluronic acid, gelatin, alginate, carboxymethylcellulose (CMC), guar gum, gellan gum, agarose and the gelatinous protein mixture secreted by Engelbreth-Holm-Swarm (EHS) mouse sarcoma cells (commercially available from Corning Life Sciences, Millipore Sigma and BD Biosciences as Matrigel® and from Trevigen, Inc. Cultrex® BME), de-cellularized patient extracellular matrix (which contains extracellular matrix components such as collagen, fibronectin, laminin, glycosaminoglycans, and other biological molecules including growth factors), PEG, hydroxyapatite, chitosan, as well as mixtures thereof. The hydrogel may also comprise polymers and copolymers with an abundance of hydrophilic group such as polyvinyl alcohol polymers and copolymers, sodium polyacrylate polymers and copolymers, acrylate polymers and copolymers, or any combination thereof.

Preferred hydrogels comprise a mixture alginate and gelatin, preferably the hydrogel comprises about 0.5% to about 3% alginate and about 5-10% gelatin, for example about 0.5% to about 2% alginate and about 6-8% gelatin, or about 1% alginate and about 7% gelatin.

The second type of cells present within the hydrogel may be any type of cells (or combination of cells) of interest, e.g., any type of cells whose interaction with and/or migration into the first type of cells bound to the matrix support is to be assessed. The second type of cells may be primary cells or a cell line, malignant or non-malignant (normal) cells, or any combination thereof. In an embodiment, the second type of cells are tumor or malignant cells, primary tumor cells or a tumor cell line, preferably a solid tumor. In an embodiment, the tumor cells are non-metastatic, i.e. have no metastasis potential. In another embodiment, the tumor cells are metastatic.

In embodiments, the tumor cell is a heart sarcoma cell, lung cancer cell, small cell lung cancer (SCLC) cell, non-small cell lung cancer (NSCLC) cell, bronchogenic carcinoma cell (squamous cell, undifferentiated small cell, undifferentiated large cell, adenocarcinoma), alveolar (bronchiolar) carcinoma cell, bronchial adenoma cell, sarcoma cell (e.g., Ewing's sarcoma, Karposi's sarcoma), chondromatous hamartoma cell, mesothelioma cell; cancer cell of the gastrointestinal system, for example, esophagus cancer cell (squamous cell carcinoma, adenocarcinoma, leiomyosarcoma, lymphoma), stomach cancer cell (carcinoma, lymphoma, leiomyosarcoma), gastric cancer cell, pancreas cancer cell (ductal adenocarcinoma, insulinoma, glucagonoma, gastrinoma, carcinoid tumors, vipoma), small bowel cancer cell (adenocarcinoma, lymphoma, carcinoid tumors, Karposi's sarcoma, leiomyoma, hemangioma, lipoma, neurofibroma, fibroma), large bowel cancer cell (adenocarcinoma, tubular adenoma, villous adenoma, hamartoma, leiomyoma); cancer cell of the genitourinary tract, for example, kidney cancer cell (adenocarcinoma, Wilm's tumor cell [nephroblastoma], lymphoma), bladder cancer cell and/or urethra cancer cell (squamous cell carcinoma, transitional cell carcinoma, adenocarcinoma), prostate cancer cell (adenocarcinoma, sarcoma), testis cancer cell (seminoma, teratoma, embryonal carcinoma, teratocarcinoma, choriocarcinoma, sarcoma, interstitial cell carcinoma, fibroma, fibroadenoma, adenomatoid tumors, lipoma); liver cancer cell, for example, hepatoma (hepatocellular carcinoma, HCC), cholangiocarcinoma, hepatoblastoma, angiosarcoma, hepatocellular adenoma, hemangioma, pancreatic endocrine tumors (such as pheochromocytoma, insulinoma, vasoactive intestinal peptide tumor, islet cell tumor and glucagonoma); bone cancer cell, for example, osteogenic sarcoma (osteosarcoma), fibrosarcoma, malignant fibrous histiocytoma, chondrosarcoma, malignant lymphoma (reticulum cell sarcoma), multiple myeloma, malignant giant cell tumor chordoma, osteochronfroma (osteocartilaginous exostoses), benign chondroma, chondroblastoma, chondromyxofibroma, osteoid osteoma and giant cell tumors; cancer cell of the nervous system, for example, neoplasms of the central nervous system (CNS), primary CNS lymphoma, skull cancer (osteoma, hemangioma, granuloma, xanthoma, osteitis deformans), meninges (meningioma, meningiosarcoma, gliomatosis), brain cancer cell (astrocytoma, medulloblastoma, glioma, ependymoma, germinoma [pinealoma], glioblastoma multiform, oligodendroglioma, schwannoma, retinoblastoma, congenital tumors), spinal cord neurofibroma, meningioma, glioma, sarcoma); cancer cell of the reproductive system, for example, gynecological cancer cell, uterine cancer cell (endometrial carcinoma), cervical cancer cell (cervical carcinoma, pre-tumor cervical dysplasia), ovarian cancer cell (ovarian carcinoma [serous cystadenocarcinoma, mucinous cystadenocarcinoma, unclassified carcinoma], granulosa-thecal cell tumors, Sertoli-Leydig cell tumors, dysgerminoma, malignant teratoma), vulvar cancer cell (squamous cell carcinoma, intraepithelial carcinoma, adenocarcinoma, fibrosarcoma, melanoma), vaginal cancer cell (clear cell carcinoma, squamous cell carcinoma, botryoid sarcoma (embryonal rhabdomyosarcoma), fallopian tube cancer cell (carcinoma); placenta cancer cell, penile cancer cell, prostate cancer cell, testicular cancer cell; cancer cell of the oral cavity, for example, lip cancer cell, tongue cancer cell, gum cancer cell, palate cancer cell, oropharynx cancer cell, nasopharynx cancer cell, sinus cancer cell; skin cancer cell, for example, malignant melanoma, cutaneous melanoma, basal cell carcinoma, squamous cell carcinoma, Karposi's sarcoma, moles dysplastic nevi, lipoma, angioma, dermatofibroma, and keloids; adrenal gland cancer cell: neuroblastoma; and cancer cells of other tissues including connective and soft tissue tumors, retroperitoneum and peritoneum, eye cancer cell, intraocular melanoma, and adnexa, breast cancer cell (e.g., ductal breast cancer), head or/and neck cancer cell (head and neck squamous cell carcinoma), anal cancer cell, thyroid cancer cell, and parathyroid cancer cell. In an embodiment the second type of cells comprise multiple types of cancer cells. In another embodiment, the second type of cells comprise at least one type of cancer cells and at least one type of non-cancer cells. In an embodiment, the non-cancer cells are cells found in a tumor, i.e. cells present in a tumor but that are not the malignant cancer cells per se (sometimes referred to as tumor-associated cells), including cancer-associated fibroblasts (CAFs), tumor-associated immune cells such as tumor-associated macrophages (TAMs) and tumor-infiltrating lymphocytes (TIFs). The second type of cells may also include any other cells, such as the cells defined above, e.g., connective tissue cells (e.g., stromal cells, fibroblasts), endothelial cells, epithelial cells, neuroglial cells, neurones, muscle cells (e.g., skeletal, cardiac, or smooth muscle cells), cartilage cells (chondrocytes), bone cells (osteoblasts, osteoclasts, osteocytes, lining cells), skin cells (keratinocytes, melanocytes, Langerhans cells), immune cells (e.g., innate immune cells such as basophils, dendritic cells, eosinophils, Langerhans cells, mast cells, monocytes and macrophages, neutrophils and NK cells, or adaptive immune cells such as B cells and T cells), astrocytes or any combination thereof, the list of course not being exhaustive.

The amount of the second type of cells incorporated in the hydrogel is selected based on various factors, including the type of cell, the size of the hydrogel and/or of the polymeric support. The number of cells may be, e.g., at least 10² or 10³ cells and up to 10⁸ or 10⁹ cells, from 10³ to 10⁷, from 10³ to 10⁶, or from 10⁴ to 10⁵ cells. In an embodiment, the cell density in the hydrogel is from about 10² to about 10⁷ cells/cm³, about 10³ to about 10⁷ cells/cm³, about 10⁴ to about 10⁶ cells/cm³, or about 10⁵ to about 10⁶ cells/cm³.

In addition to the components defined above, the second layer (hydrogel) may further comprise other materials such as extracellular matrix molecules, proteins, peptides, nucleic acids, dyes (fluorescent dyes), etc.

In an embodiment, the 3D cell culture system further comprises a culture medium. The culture medium may be selected based on the first and/or second type of cells, i.e. to allow the growth of the cells. Such culture medium are well known in the art, and include, e.g., MEM, DMEM, EMEM, IMDM, RPMI 1640, Ham's F12, Ham's F10, media for endothelial cell such as human Endothelial-SFM (Life Technologies), Endothelial Basal Media, EndoGRO-LS Complete Media Kit (MilliporeSigma), HUVEC Basal Medium CB HUVEC (AllCells), and Endothelial Cell Medium (ScienCell Research Laboratories), media for glial cell such as GIBCO® Astrocyte Medium, media for bone marrow cells such as MarrowMAX Bone Marrow Medium (Life Technologies) and Bone Marrow Medium Plus (MilliporeSigma), media for epithelial cells such as Epithelial cell medium (ScienCell Research Laboratory), EpiGRO primary epithelial cells (MilliporeSigma), media for T cells such as Human StemXVivo Serum-Free T cell Base Media (R&D systems), Stemline T cell Expansion Medium (MilliporeSigma), and media for hematopoietic stem cells such as StemPro-34 SFM (Life Technologies) and MethoCult (STEMCELL Technologies, Inc). These media may be supplemented with nutrients, serum, antibiotics, growth factors, cytokines, etc. as appropriate.

In embodiments, the 3D cell culture system further comprises differentiating or growth factors such as a bone morphogenetic protein, a cartilage-derived morphogenic protein, a growth differentiation factor, an angiogenic factor, a platelet-derived growth factor, a vascular endothelial growth factor, an epidermal growth factor, a fibroblast growth factor, a hepatocyte growth factor, an insulin-like growth factor, a nerve growth factor, a colony-stimulating factor, a neurotrophin, a growth hormone, an interleukin, a connective tissue growth factor, a parathyroid hormone-related protein, etc. Such differentiating or growth factors may be added at any time during cell culture to stimulate the growth and/or differentiation of the first and/or second type of cells as desired.

In an embodiment, the 3D cell culture system further comprises one or more additional layers, e.g., a third layer or a third and a fourth layer. The one or more additional layers may be under, between or over (on top of) the first and second layers. The one or more additional layers may comprise the same components as the first or second layer, or different components (e.g., a third type of cells). In an embodiment, the 3D cell culture system further comprises a third layer, wherein the third layer comprises the same components as the first or second layer. In a further embodiment, the third layer comprises the same components as the first layer. In yet a further embodiment, the third layer is over the first and second layers.

The first and/second type of cells present in the 3D cell culture system described herein may form different shapes such as aggregates, spheroids, tumoroids or organoids.

In an embodiment, the 3D cell culture system is in a container or device, for example a cell culture plate, such as a 6-well plate, a 12-well plate, a 24-well plate, a 96-well plate, a 384-well plate, a cell culture dish, a cell culture flask, e.g., a multi-layer flask, etc. a bioreactor. In an embodiment, the container is a multi-well plate for high throughput screening (HTS).

### Method of Preparing the 3D Cell Culture System

The present disclosure also provides a method for preparing a three-dimensional (3D) cell culture system, the method comprising:
(i) providing a functionalized solid porous matrix support made of a preferably biocompatible material, such as a polymer;
(ii) seeding a first type of cells on the functionalized solid porous matrix support to attach the first cell type on the solid porous matrix support;
(iii) contacting the solid porous polymeric support of step (ii) with a biocompatible hydrogel comprising a second type of cells, thereby obtaining the 3D cell culture system.

In an embodiment, the method further comprises, prior to step (ii), submitting the solid porous matrix support to plasma treatment to obtain the functionalized solid porous matrix support. The plasma treatment may be performed using any of the methods described above, e.g., by plasma-induced grafting (surface modification) or by plasma-enhanced chemical vapor deposition (PECVD). In an embodiment, the method further comprises, prior to step (i), preparing the polymeric support, e.g., by electrospinning of non-woven materials to obtain nanofibers and/or microfibers, or by 3D printing.

The method may further comprise one or more additional steps, such as a step of culturing the 3D cell culture system under suitable conditions, e.g., to allow survival, growth, differentiation of the first and/or second type of cells. In an embodiment, the second type of cells migrate at the surface and/or into the solid porous polymeric support during said culturing. This culturing step is performed for a sufficient time to allow, e.g., cell growth, differentiation and/or migration from the hydrogel to the surface and/or into the solid porous polymeric support, e.g., for at least 1 day, 2 days, 3 days, 1 week, 2 weeks, 3 weeks, 1 month, 2 months, or 3 months.

### Uses of 3D Cell Culture System

The present disclosure relates to the use of the 3D cell culture system described herein for the identification and/or evaluation of cell activity, function or behavior, including but not limited to differentiation, response to toxic chemicals (e.g. metals ions, drugs, therapeutics) or co-cultures (e.g., cancer cells, immune cells, fibroblasts).

The 3D cell culture system described herein may be used for various applications including regenerative medicine, tissue engineering, screening compounds for biological use, or drug screening.

Also provided is a method of testing or screening a candidate compound or agent using the 3D cell culture system described herein. The 3D cell culture system may be used for assessing the effect of the candidate compound or agent on the first and/or second type of cells, e.g., on the growth, survival, function (e.g., gene expression), and/or migration of the first and/or second type of cells.

In an embodiment, the first and/or second types of cells comprise tumor cells, and the method described comprises testing or screening a candidate compound or agent on carcinogenesis or for its effect on cancer tissue, and comprises contacting the 3D cell culture system described herein with the candidate compound or agent or contacting the 3D cell culture system described herein with the candidate compound or agent and maintaining said contacted 3D cell culture system in culture, and observing any changes in the 3D cell culture system relative to said 3D cell culture system without contacting by said candidate compound.

Likewise, the present disclosure provides exposing the 3D cell culture system to a condition instead of contacting it with a candidate compound. Such a condition may be e.g., elevated temperature, electromagnetic radiation, sound waves, electrical stimulation, mechanical force, limited nutrients, radiations or altered redox potential, to which cells such as cancer cells may react and exhibit a different behavior or growth rate as compared to behavior or growth without exposure to said condition. Accordingly, the 3D cell culture and the method of its generation can also be used as a research tool to study the effects of any chemical (compounds, e.g. drugs or other stimuli), (biological) agents (e.g. a virus, like an oncolytic virus and/or a Flavivirus) environmental (e.g., temperature, pressure, light exposure, redox potential, nutrients, irradiation) influences on growth, survival, function, and/or migration of cells in the 3D cell culture system, in particular of the cells undergoing carcinogenesis. Temperature changes are preferably elevated temperature; altered nutrients are, e.g., lowered glucose or other carbohydrate energy sources, increased fat or fatty acids; altered redox potential may be, e.g., the addition of oxidizing agents or reducing agents or antioxidants, like vitamin C; light may be UV light; irradiation may be by alpha or beta radiation sources; a virus may be an oncolytic virus. It is further possible to compare the effects on the cancer cells to the effects on the non-cancerous cells of the same or a different 3D cell culture system. Accordingly, it is possible to identify cancer specific compounds, agents or environmental factors that have a stronger effect on cancer cells than non-cancer cells. In this case, compounds, agents or environmental factors may be eligible cancer therapy candidates, vs. compounds or agents or environmental factors that kill cancerous and noncancerous cells indiscriminately.

The candidate compound or agent may be analyzed and selected according to a desired property on the development of cancer in the 3D cell culture system. For example, compounds or agents may be analyzed for their potential to slow or even halt cancer growth, for their ability to stimulate immune cells present in the tumor, for their ability to destroy tumor or cancer cells, and/or for their ability to inhibit the migration of tumor or cancer cells (e.g., metastasis). Such effects can be screened in comparison to the non-cancerous cells, which are preferably less affected by such detrimental effects than the cancer cells, if the candidate compound should be further considered as a cancer treatment drug. Any kind of activity of the 3D cell culture system, including metabolic turn-over or signaling can be searched for in a candidate compound or agent. In essence, the 3D cell culture system can be used as a model for tissue behavior testing on any effects of any compound. Such a method might also be used to test therapeutic drugs, intended for treating cancer, for having side-effects on non-cancerous cells as can be observed in the 3D cell culture system. As said, instead of testing or screening a candidate compound or agent, also environmental conditions can be analyzed for the same effects and purposes. Such effects may be elevated temperatures, such as 40 °C and above, or reduced nutrients like withdrawal of a carbohydrate or mineral source.

The 3D cell culture system described herein could also be used for screening agents (e.g., candidate compounds, bioactive molecules) on tissue repair/regeneration. By selecting the first and second types of cells to mimic a tissue of interest, it is possible to assess whether an agent stimulates or inhibits repair and/or regeneration of the tissue of interest. For example, for assessing the effect of an agent on cartilage repair, a 3D cell culture system comprising bone cells in the functionalized solid porous matrix support, and chondrocytes embedded in the hydrogel, could be used.

A candidate drug as candidate compound or agent may be a biomolecule, like a protein (e.g., antibody), peptide, nucleic acid, or comprise or be composed of such biomolecules, such as a virus, or a small molecule inhibitor. Small molecules are usually small organic compounds having a size of 5000 Dalton or less, e.g., 2500 Dalton or less, or even 1000 Dalton or less. The candidate drug, agent or compound may be known for other indication and/or a known chemical compound. Such known compounds are, e.g., disclosed in compound databases such as Selleckchem (www.selleckchem.com), which collects inhibitor compound information, including the cellular target of a compound. In some embodiments, the candidate agent is a cell. Therapeutic cells are known in the art and include stem cells, progenitor cells, and immune cell. The cells can be isolated cells, cell lines, or engineered cells (e.g., chimeric antigen receptor (CAR) cell such as CAR-T cells or CAR-NK cells).

The effect of the candidate agents and stimuli on cells may be evaluated from a sample collected from the 3D cell culture. The method may thus also comprise performing one or more tests on the cells (e.g., on a sample from the 3D cell culture) before, during and/or after the cell culture, such as imaging the cells, measuring the presence/level of markers, assessing the number of cells in one or more of the layers, assessing genomic alterations in the cells, assessing gene/protein expression, assessing the production of metabolites by cells, etc. Thus, the cell may be analyzed by an immunoassay such as enzyme linked immunosorbent assay (ELISA), radioimmunoassay (RIA), radioimmune precipitation assay (RIPA), immunobead capture assay, Western blotting, dot blotting, gel-shift assay, flow cytometry, protein array, multiplexed bead array, magnetic capture, imaging, fluorescence or bioluminescence resonance energy transfer (FRET/BRET), and fluorescence recovery/localization after photobleaching (FRAP/FLAP), or by a gene expression assay such as Northern blot, RNAse protection assay, reverse transcription (RT)-PCR, real-time PGR (qPCR), in-situ hybridization, dot-blot analysis, differential display, subtractive hybridization, DNA microarray, RNA microarray, NANOSTRING, and next generation sequencing (NGS).

The effect of the candidate agents and stimuli may be assessed in an extracellular microenvironment sample. For example, the extracellular microenvironment may be analyzed for the presence of a protein, nucleic acid, lipid, carbohydrate, or any combination thereof. In some embodiments, the extracellular microenvironment is analyzed for pH, gases, salts, or other such physical, biological, and/or chemical properties.

The method may further involve imaging the cells before, during or after the culture period. For example, the cells can be imaged continuously during culture. In some embodiments, the method comprises the use of a system comprising a computer capable of analyzing the images and tracking the cells in the culture. This can be useful in evaluating, for example, cell growth, shape, motility, interaction, migration, etc.

### DEFINITIONS

The use of the terms “a” and “an” and “the” and similar referents in the context of describing the invention (especially in the context of the following claims) are to be construed to cover both the singular and the plural, unless otherwise indicated herein or clearly contradicted by context.

The terms “comprising”, “having”, “including”, and “containing” are to be construed as open-ended terms (i.e., meaning “including, but not limited to”) unless otherwise noted.

Recitation of ranges of values herein are merely intended to serve as a shorthand method of referring individually to each separate value falling within the range, unless otherwise indicated herein, and each separate value is incorporated into the specification as if it were individually recited herein. All subsets of values within the ranges are also incorporated into the specification as if they were individually recited herein.

All methods described herein can be performed in any suitable order unless otherwise indicated herein or otherwise clearly contradicted by context.

The use of any and all examples, or exemplary language (e.g., “such as”) provided herein, is intended merely to better illuminate the invention and does not pose a limitation on the scope of the invention unless otherwise claimed.

No language in the specification should be construed as indicating any non-claimed element as essential to the practice of the invention.

Herein, the term “about” has its ordinary meaning. In embodiments, it means plus or minus 5% of the numerical value qualified.

Unless otherwise defined, all technical and scientific terms used herein have the same meaning as commonly understood by one of ordinary skill in the art to which this invention belongs.

Other objects, advantages and features of the present invention will become more apparent upon reading of the following non-restrictive description of specific embodiments thereof, given by way of example only with reference to the accompanying drawings.

### DESCRIPTION OF ILLUSTRATIVE EMBODIMENTS

The present invention is illustrated in further details by the following non-limiting examples.

### Example 1: Preparation and Testing of 3D Electrospun PLA Scaffolds

A medium size PLA mat was prepared by electrospinning using the following method. A 16 wt % PLA solution was prepared by dissolving PLA pellets (Ingeo™ Biopolymer 4032D, NatureWorks) in 2,2,2 Trifluoroethanol (TFE) and stirring for 24 h. By using a syringe pump placed in a chamber with a controlled temperature (21-25 °C) and relative humidity of about 45%-50%, the polymer solution was electrospun with a flow rate of 1.6 ml/hr. The distance of the grounded needle tip (21 G) and a rotating collector (25 rpm) was set at 15 cm and the applied voltage of 20 kv between the needle tip and the rotating mandrel was provided constantly by a power supply. The large size PLA electrospun mat was prepared using the following processing parameters: 19 wt % PLA in TFE solution, needle tip of 18 G, distance of 15 cm, flow rate of 1.5 ml/hr and voltage of 20 kv. The small size PLA electrospun mat was prepared using the following processing parameters: 14 wt % PLA and 0.1 wt % NaCl in TFE solution, needle tip of 26 G, distance of 20 cm, flow rate of 0.7 ml/hr and voltage of 22 kv.

The surface of the PLA mat scaffold was treated with oxygen gas in a low pressure (610 millitorr) glow discharge plasma reactor with a flow rate of 15 standard cubic centimeters per minute (sccm) for a duration of 30 sec and under mild plasma condition (power: 15 W and Voltage: −40V). NH₃ plasma treatment was also performed on the surface of PLA solid scaffolds as the flow rate of ammonia gas was 15 sccm with the exposure time of 1 min for both sides. Furthermore, plasma coating (L-PPE:N) was deposited only on the surface of PLA scaffolds with the gas mixture of ethylene (C₂H₄) and ammonia (NH₃) with flow rate of 20 and 15 sccm, respectively and deposition time of 7.5 min for both sides. PLA mat scaffolds that were not plasma-treated were also prepared as control.

50,000 cells (breast cancer cells or osteoblast cells) were seeded onto the PLA mats. Mats comprising three different (average) fiber diameters were investigated, namely “small” (200-400 nm); “medium” (600-800 nm); and “large” (1.0-2.0 μm) using different PLA solutions (14 wt %, 16 wt % and 19 wt % PLA solution, respectively). Tables 1 and 2 show the overall porosity percentage and average pore size, respectively, of PLA electrospun mats in small, medium and large sizes.

TABLE 1
Overall porosity percentage of PLA electrospun
mats in small, medium and large sizes.

| Scaffold | W1 (mg) | W2 (mg) | Porosity (%) |
|---|---|---|---|
| Large | 33.96 | 324.5 | 89.5 |
| Medium | 21.5 | 241.9 | 91.1 |
| Small | 26.1 | 363.1 | 92.7 |

Porosity properties of nanofiber mats were determined using a liquid (ethanol) intrusion method. Dry mats were weighed before being immersed in 100% ethanol overnight for complete wetting (W1). Mats were then gently wiped to remove excess ethanol and weighed again (W2). Porosity is defined as the volume of the ethanol entrapped in the pores divided by the total volume of the wet mats (ethanol.mat). % Porosity=(W2−W1)/W2*100

TABLE 2
Average pore size of PLA electrospun mats
in small, medium and large sizes.

| Scaffold | Particle count | S.D. | Average Pore Size (μm) |
|---|---|---|---|
| Large | 300 | 18.41 | 6.2 |
| Medium | 250 | 24 | 3.1 |
| Small | 425 | 6.3 | 0.5 |

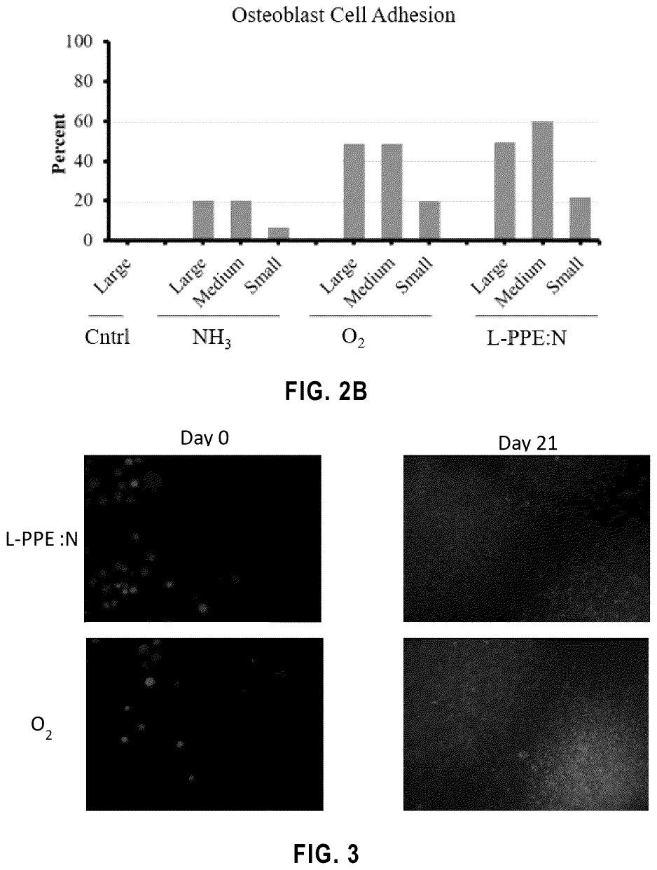

The results are presented in FIGS. 2A and 2B. The efficacy of plasma surface modification, either by treatment or by PP coating, is clearly evident from the much higher proportion of breast cancer cells (FIG. 2A) and osteoblasts (FIG. 2B) that were found adhering within the mats 24 hours after seeding, relative to controls (“Cntrl”). Significant cell adhesion was obtained for mats comprising small, medium and large average fiber diameters.

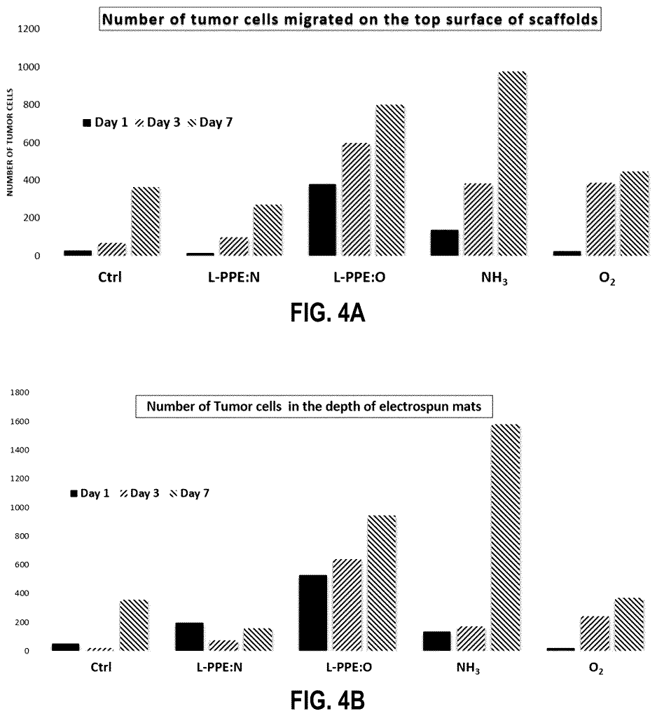

FIG. 3 shows the following particular embodiment of this technology: in this example, the 3D matrix was seeded with fibroblastic tumor cells, while the hydrogel contained breast cancer cells. Two different plasma treatments (O₂ plasma surface modification; and L-PPE:N coating) was carried out on two separate batches of electrospun mats and the images represent a sample from each of those two batches. It was observed that after 21 days of culture, the breast cancer cells had migrated from the hydrogel and proliferated in the 3D electrospun scaffolds, where they are seen to have displaced the fibroblasts. Both types of plasma treatments led to comparable results.

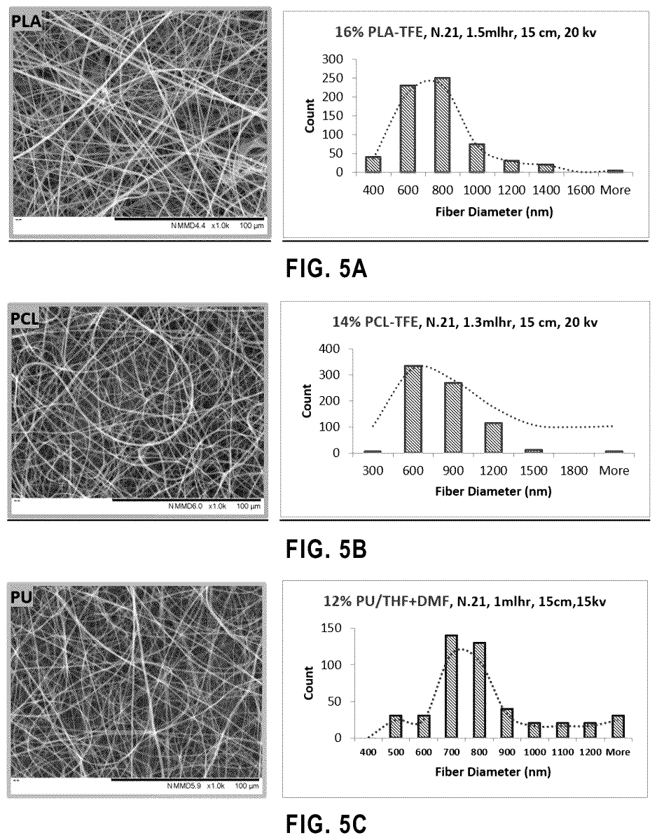

The effects of various plasma treatments on tumor cell migration was also tested. 20,000 fibroblasts were seeded on PLA scaffolds functionalized with the following treatments: O₂ plasma surface modification; NH₃ plasma surface modification, L-PPE:N coating, and L-PPE:O coating. 20,000 MDA-MB-231 breast cancer cells in hydrogel were seeded on the functionalized PLA scaffolds, and the migration of the tumor cells at the surface and inside the scaffolds was assessed at day 1, day 3 and day 7. The results are depicted in FIG. 4A (surface) and FIG. 4B (inside).

### Example 2: Preparation and Testing of Other 3D Biocompatible Polymeric Scaffolds

It was next assessed whether solid scaffolds made of various biocompatible polymers, as well as 3D printed scaffolds, were suitable to prepare the 3D cell culture system.

### Materials and Methods

### 1. Scaffold Preparation

### 1.1 Electrospinning

A) Medium size PLA electrospun mat: 16 wt % PLA solution was prepared by dissolving PLA pellets in 2,2,2 Trifluoroethanol (TFE) and stirring for 24 h. By using a syringe pump placed in a chamber with a controlled temperature (21-25 °C) and relative humidity of about 45%-50%, the polymer solution was electrospun with a flow rate of 1.6 ml/hr. The distance of the grounded needle tip (21 G) and a rotating collector (25 rpm) was set at 15 cm and the applied voltage of 20 kv between the needle tip and the rotating mandrel was provided constantly by a power supply.
B) PCL electrospun mat: 14 wt % PCL solution prepared by dissolving PCL pellets (Millipore Sigma, Cat No. 440744) in 2,2,2 Trifluoroethanol (TFE) was used. The parameters were the same as A), except that the flow rate was 1.3 ml/hr.
C) PU electrospun mat: 12 wt % PU solution prepared by dissolving PU pellets (Millipore Sigma, Cat. No. 430218) in 1:1 mixture of Tetrahydrofuran (THF) and Dimethylformamide (DMF). The parameters were the same as A), except that the flow rate was 1.0 ml/hr and the applied voltage was 15 kv.

The thickness of the scaffolds was adjusted to 200-250 m.

### 1.2 3D Printing Technique

By using a 3D desktop printer, 3D microporous cuboidal PLA scaffolds with pore sizes of 750 m (medium) and overall dimensions of 10 mm×10 mm×4 mm were prepared. The filament of PLA was extruded at melting temperature of 220 °C from a 0.3 mm nozzle with printing time of 45 min for medium microporous scaffolds.

### 2. Plasma Treatment

The surface of nanofibrous electrospun mats and 3D-printed scaffolds were treated with oxygen gas in a low pressure (610 millitorr) glow discharge plasma reactor with a flow rate of 15 standard cubic centimeters per minute (sccm) for a duration of 30 sec and under mild plasma condition (power: 15 W and self-bias Voltage: −40V). Furthermore, plasma coating (“L-PPE:N”) was deposited only on the surface of PLA 3D-printed scaffolds with the gas mixture of ethylene (C₂H₄) and ammonia (NH₃) with flow rate of 20 and 15 sccm, respectively and deposition time of 7.5 min for both sides. NH₃ plasma treatment was also performed on the surface of PLA solid scaffolds as the flow rate of ammonia gas was 15 sccm with the exposure time of 1 min for both sides.

Ethyl lactate (PP-EL) and allylamine (PP-AAm) plasma polymer coatings were prepared at ca. 100 kPa pressure in dielectric barrier discharge (DBD) plasmas using a mixture of 10 standard liters per minute (slm) of pure argon (Ar) carrier gas into which is mixed a few standard cubic centimeters per minute (sccm) of the monomer vapor, ethyl lactate (EL) and allyl amine (AAm), respectively, all this using audio-frequency (AF, ca. 20 kHz) high-voltage (8 kV peak-to-peak) electric power from a suitable dedicated power supply.

### 3. Cell Culture and Seeding

### A) Electrospun Scaffold

i) Plasma-treated scaffolds punched in 9 mm-disks were sterilized by media containing antibiotic (RPMI 1640 with 10% FBS and 1% Penstrep) and fitted into a non-stick 48-well plate, quadruplicate.
ii) Stromal cell line for seeding on sterilized scaffolds: Malignant Fibroblast RFP; passage #5; 20,000 cells/scaffold.
iii) Epithelial breast cancer cell line: MDA-MB231 GFP, passage #35; encapsulated in hydrogel A1G7 (1% alginate, 7% gelatin); 10,000 cells/scaffold on top of the pre-seeded scaffolds.
iv) Monitoring of the system at day 0, 1, 3 and 7.

### B) 3D Printed Scaffold

i) Treated scaffolds were washed in media containing antibiotic (DMEM with 10% FBS and 0.5% Gentamicin) before seeding with cells.
ii) Cell line for seeding: human Dental Pulp Stem Cells (hDPSCs), Passage #4; 500,000 cells for PLA scaffold, 200,000 cells for PCL and PU scaffolds.

### Results

### 1. Evaluation of Nanofibrous Scaffold Morphology

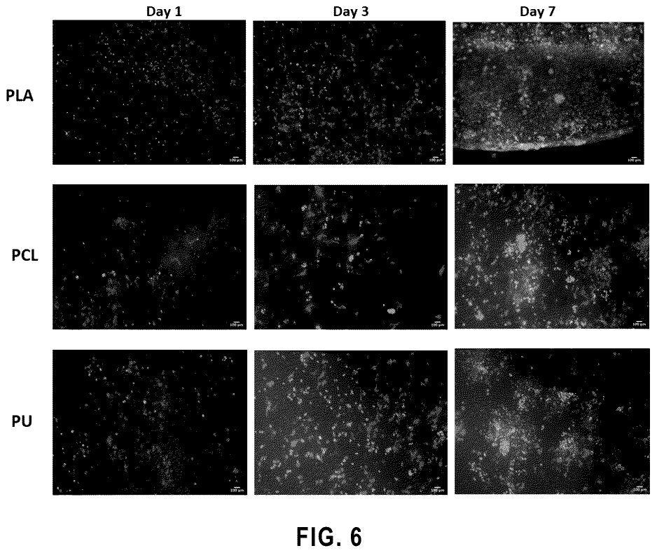

The surface morphology of PCL and PU nanofiber scaffolds were characterized by Scanning Electron Microscopy (SEM) and the value of fiber diameter obtained by micrographs was found to be in the range from 600-800 nm (medium size), similar to PLA electrospun mats fabricated in pervious experiment (FIGS. 5A-C).

### 2. Observation of Cellular Adhesion and Tumor Migration on Nanofibrous Scaffolds

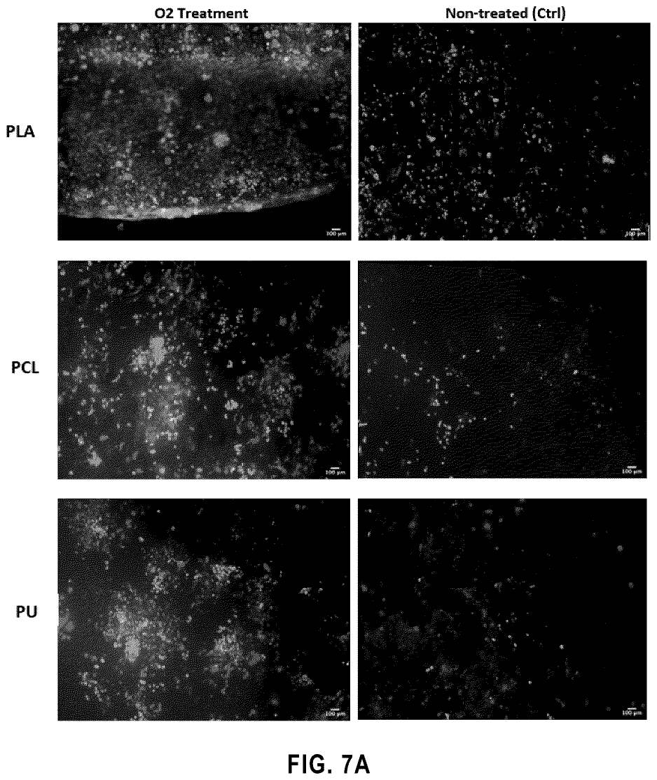

By using laser confocal microscopy, the top surface of PCL and PU scaffolds, non-treated and treated with O₂ plasma were monitored at different time points. The images depicted in FIG. 6 show that tumor cells proliferate and migrate on the surface of treated PU and PCL scaffolds at days 1, 3 and 7, similar to treated PLA scaffolds. It was confirmed that the pattern of tumor migration for PCL and PU was comparable to that for PLA, in which the number of tumor cells migrated on the surface of scaffold increased over the time.

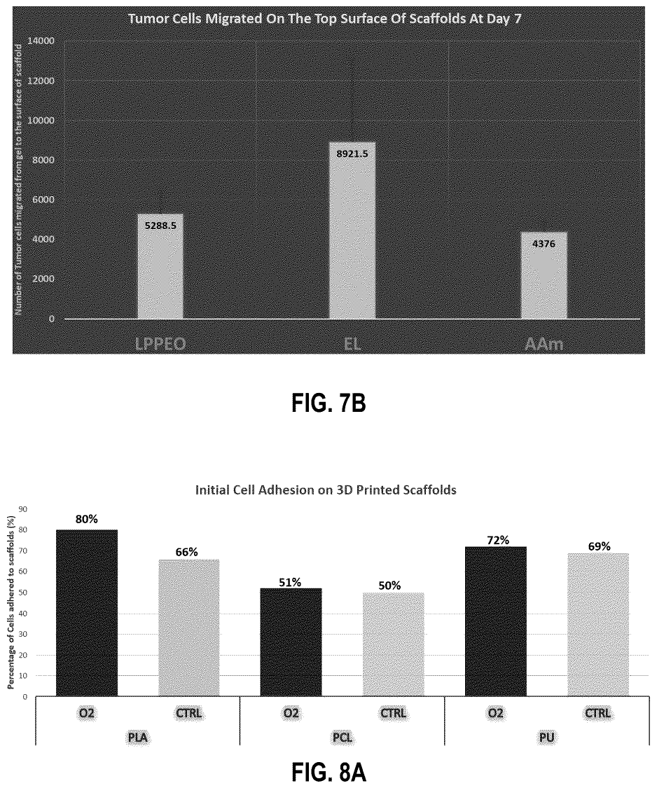

In addition, FIG. 7A shows that for PLA, PCL and PU scaffolds, the amount of tumor proliferation and/or migration at day 7 on the samples treated with O₂ plasma was significantly more than on non-treated electrospun mats.

The results depicted in FIG. 7B show the migration of MDA-MB 231 breast cancer cells from the hydrogel to the top surface of PLA electrospun mats (medium size) treated with three different plasma coatings including L-PPE:O from ethylene+oxygen mixture, and PP-EL and PP-AAm from the monomers ethyl lactate (EL), and allylamine (AAm), respectively.

### 3. Initial Cell Adhesion and Proliferation Observation on 3D Printed Scaffolds

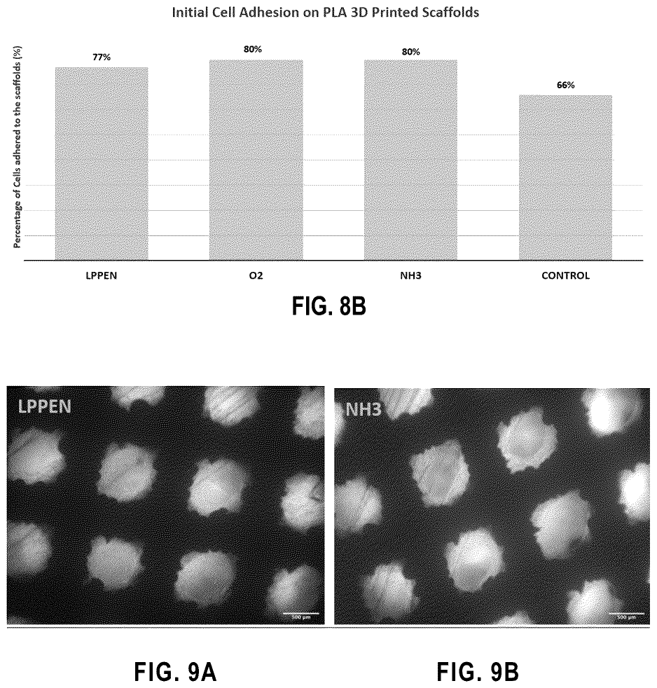

3D printed solid scaffolds based on PLA, PCL and PU were seeded with human dental stem cells. Initial cell adhesion on O₂ plasma treated and non-treated scaffolds was measured, and the results are shown in FIG. 8A. The results depicted in FIG. 8B show cell adhesion in 3D printed PLA scaffolds treated with O₂ plasma, NH₃ plasma and L-PPE:N coating, which was superior to cell adhesion in control (untreated) scaffolds. These results provide evidence that O₂ plasma, NH₃ plasma and L-PPE:N treated scaffolds of all polymeric samples promote initial cell adhesion to the surface, relative to non-treated control scaffolds.

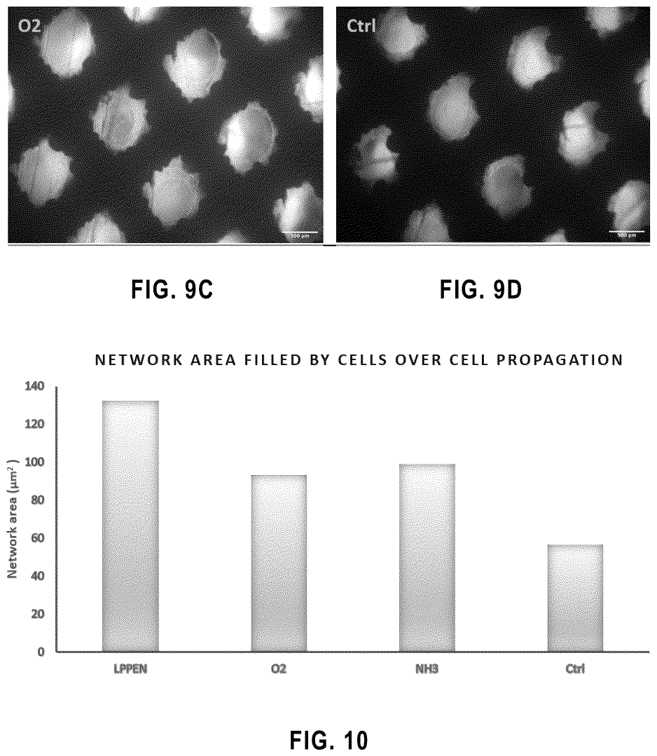

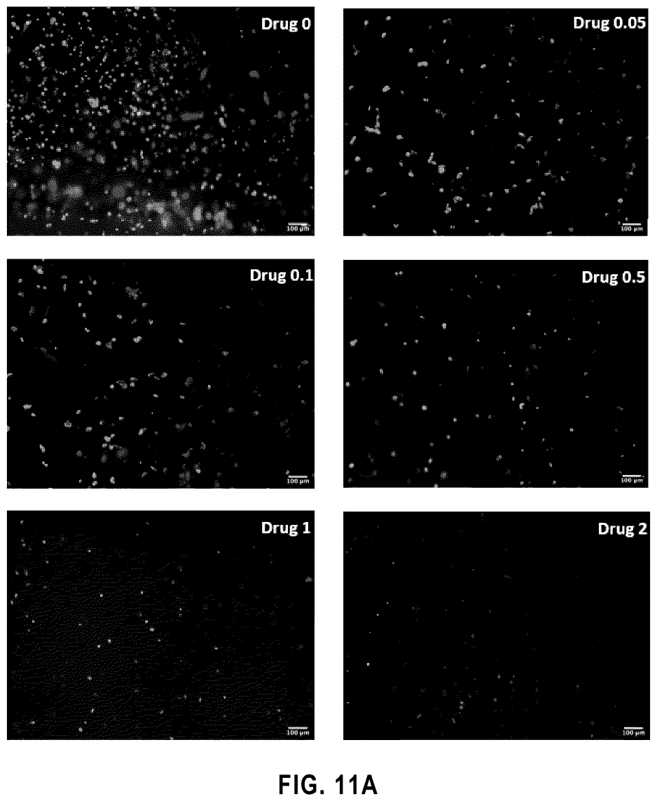

Moreover, FIGS. 9A-D show cell proliferation and growth inside treated and non-treated 3D printed scaffolds, monitored over 21 days of culture. A network of cells, along with their extracellular matrix, was clearly observed inside the pores. It is worth mentioning that the amount of cells propagation inside the pores is higher for the scaffolds coated with L-PPE:N relative to other types of treated scaffolds. For each sample, the network area produced in the pores (n=8) was calculated and the result is shown in FIG. 10. Scaffolds treated with L-PPE:N showed the greatest ECM network produced by stem cells, and also the non-treated scaffold led to the smallest area covered by cells over 21 days of cell growth.

### Example 3: Drug Screening Test on PLA Electrospun Mat

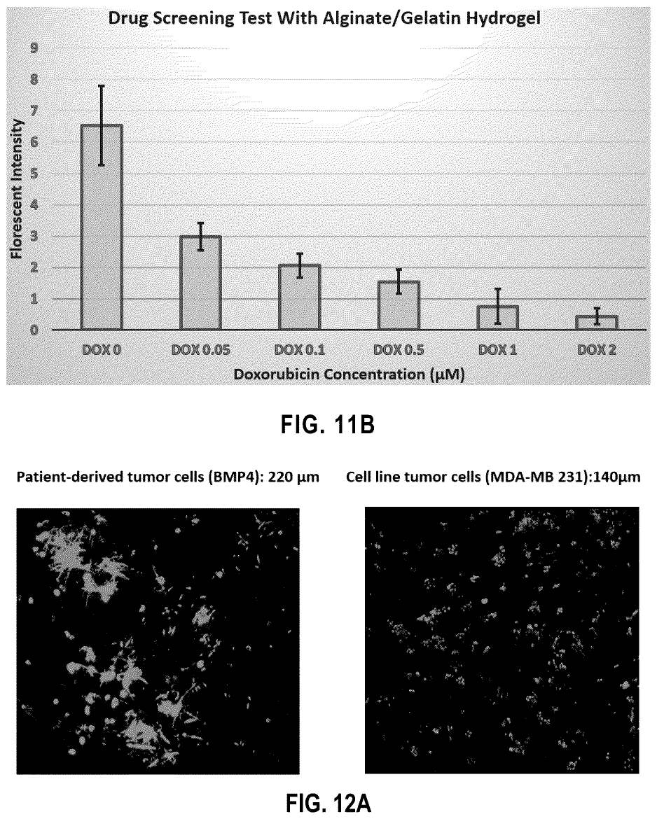

A drug screening setup was designed using the known anti-tumor drug Doxorubicin to assess tumor migration performance on the 3D cell culture model of the present disclosure. The plasma-treated PLA electrospun mats seeded with fibroblasts and tumor cells on A1G7 hydrogel on top, were loaded with various concentrations of Doxorubicin (0, 0.05, 0.1, 0.5, 1 and 2 M) in triplicate, and tumor migration assessment was performed at day 7 of cell culture. As shown in the images depicted at FIG. 11A and the corresponding graph of FIG. 11B, by increasing the amount of drug in the media, there was a significant reduction in the number of breast cancer cells that were able to migrate to the surface of scaffolds. Moreover, it seems that fibroblasts were also affected by Doxorubicin, as evidenced by the reduced number of stromal cells on nanofibrous scaffolds relative to the samples from previous experiment (e.g., in FIGS. 5 and 6). The experiment was done in triplicate and the error bars presented in the chart are based on standard deviation.

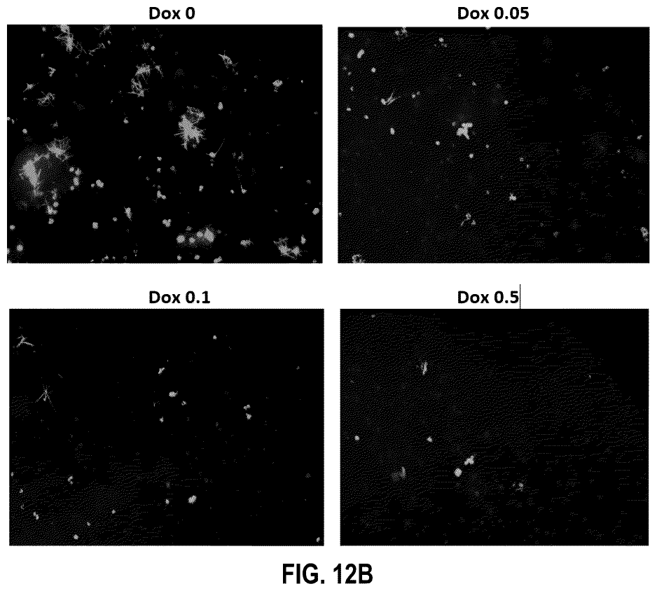

The 3D cell culture system according to the present disclosure was also used to perform a doxorubicin screening tested with tumor cells derived from patients (bone metastases prostate (BMP4) patient-derived tumor cells) and a breast tumor cell line (MBA-MB231) in A1G7 hydrogel. In this experiment, the PLA scaffolds were coated with PP-EL, ethyl lactate (EL) plasma coating in atmospheric pressure, and captured at day 7 after culture. The results depicted in FIG. 12A indicate a significant difference in the appearance of primary BMP4 tumor cells and commercial tumor cell line in the images. The pictures show the 3D stack of the total mat layers (certain thickness) obtained by signal from cells.

The PP-EL plasma-coated PLA electrospun mats seeded with BMP4 tumor cells on A1G7 hydrogel on top, were loaded with various concentrations of Doxorubicin (0, 0.05, 0.1 and 0.5 M) in triplicate, and tumor migration assessment was performed at day 7 of cell culture. A dose-dependent inhibition of BMP4 tumor cells migration was measured (FIGS. 12B and 12C)

### Example 4: Comparison of Drug Screening Test: 3D Biocompatible Polymeric Scaffolds with A1G7 Hydrogel Vs. Matrigel®

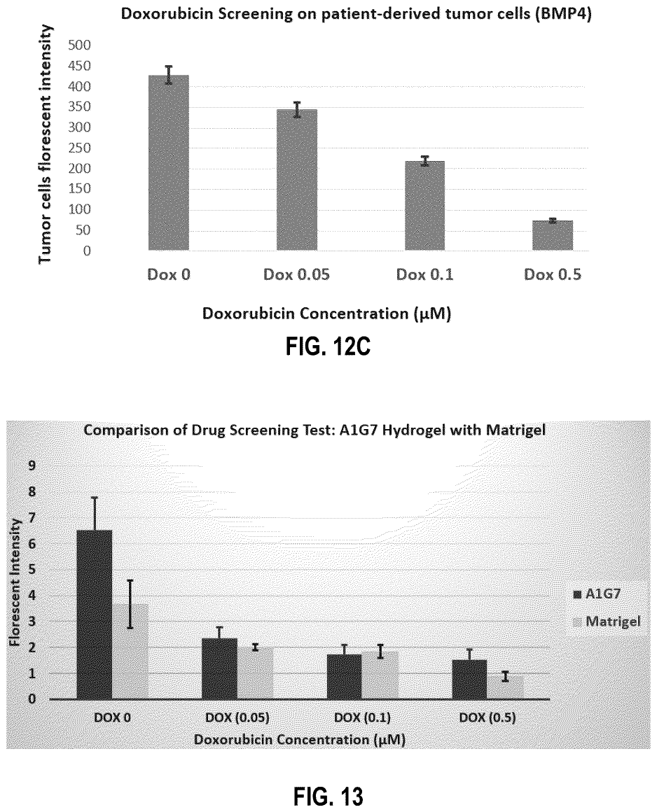

An alternative drug screening setup was also planned and considered based on Matrigel®, a well-established material for tumor metastasis assessment with the aim of comparing its result with A1G7 hydrogel according to the present disclosure. By using a 24-well plate, similar number of fibroblasts to the experiment described above (20,000) were seeded on the surface of each well of the plate while a transparent polyethylene terephthalate (PET) membrane appropriate for 24-well plate with the pore size of 8 m was placed on top loading with Matrigel®-containing breast cancer cells (MDA-MB 231), 10,000 cells for the each well of the membranes, in triplicate. Upper and lower sides of the membranes were loaded with various concentrations of Doxorubicin including 0, 0.05, 0.1, 0.5 M along with media and tumor migration investigation was performed at day 7 of cell culture. The results depicted in FIG. 13 shows that the system based on A1G7 hydrogel according to the present disclosure (left bars) is comparable to the Matrigel®-based system (right bars) to assess tumor cell migration.

The scope of the claims should not be limited by the preferred embodiments set forth in the examples, but should be given the broadest interpretation consistent with the description as a whole.

### REFERENCES

The present description refers to a number of documents, the content of which is herein incorporated by reference in their entirety. These documents include, but are not limited to, the following:
R. Wyrwa et al, “Design of Plasma Surface-Activated, Electrospun Polylactide Non-Wovens with Improved Cell Acceptance”, Advanced Engineering Materials, 13(5), B165-B171 (2011) DOI: 10.1002/adem.201080116
J. P. Chen, “Surface modification of electrospun PLLA nanofibers by plasma treatment and cationized gelatin immobilization for cartilage tissue engineering”, Acta Biomater. 2011 January; 7(1):234-43. doi: 10.1016/j.actbio.2010.08.015. Epub 2010 Aug. 20.
M. T. Nelson et al., “Preferential, enhanced breast cancer cell migration on biomimetic electrospun nanofiber ‘cell highways’” BMC Cancer 2014, 14:825
Wei Zhu et al., «Cold Atmospheric Plasma Modified Electrospun Scaffolds with Embedded Microspheres for Improved Cartilage Regeneration”, PLOS ONE | DOI:10.1371/journal.pone.0134729 (2015)
B. Delalat, D. Hutmacher et al., “3D printed lattices as an activation and expansion platform for T cell therapy”, Biomaterials 140, 58e68 (2017)
P. Liu et al., “Surface modification of porous PLGA scaffolds with plasma for preventing dimensional shrinkage and promoting scaffoldcell/tissue interactions”, Journal of Materials Chemistry B (2018)
Houman Savoji, Afra Hadjizadeh, Marion Maire, Abdellah Ajji, Michael R. Wertheimer, Sophie Lerouge (2014), “Electrospun Nanofiber Scaffolds and Plasma Polymerization: A Promising Combination Towards Complete, Stable Endothelial Lining for Vascular Grafts”, Macromol. Biosci., 14, 1084-1095
Houman Savoli, Marion Maire, Pauline Lequoy, Benoit Liberelle, Gregory De Crescenzo, Abdellah Ajji, Michael R. Wertheimer, Sophie Lerouge (2017), “Combining electrospun fiber mats and bioactive coatings for vascular graft prostheses”, Biomacromolecules, 18 (1), pp 303-310. DOI: 10.1021/acs.biomac.6b0177
P. Ahangar, E. Akoury, A. S. Ramirez Garcia Luna, A. Nour, M. H. Weber and D. H. Rosenzweig, “Nanoporous 3D-Printed Scaffolds for Local Doxorubicin Delivery in Bone Metastases Secondary to Prostate Cancer”, Materials 2018, 11, 1485; doi:10.3390/ma11091485
E. Akoury, M. H. Weber, D. H. Rosenzweig “3D-Printed Nanoporous Scaffolds Impregnated With Zoledronate For The Treatment Of Spinal Bone Metastases”, MRS Advances 3039079 2018
E. Akoury, A. S. Ramirez Garcia Luna, P. Ahangar, X. Ga, P. Zolotarov., M. H. Weber, D. H. Rosenzweig, “Low-Dose Zoledronate For Local Delivery To 2 Patient-Derived Spinal Bone Metastasis Secondary To 3 Lung Cancer”, Cancers 2019, 11.
E. Akoury, P. Ahangar, A. Nour, J. Lapointe, K.-P. Guérard, L. Haglund, D. H. Rosenzweig and M. H. Weber, “Low-Dose Zoledronate For The Treatment Of Bone Metastasis Secondary To Prostate Cancer”, Cancer Cell Int. (2019) 19:28
Chen, S., Li, R., Li, X., & Xie, J. (2018). Electrospinning: An enabling nanotechnology platform for drug delivery and regenerative medicine. Advanced drug delivery reviews, 132, 188-213.
Zhou, Y., Chyu, J., & Zumwalt, M. (2018). Recent Progress of Fabrication of Cell Scaffold by Electrospinning Technique for Articular Cartilage Tissue Engineering. International journal of biomaterials, 2018.
Chen, S., Boda, S. K., Batra, S. K., Li, X., & Xie, J. (2018). Emerging roles of electrospun nanofibers in cancer research. Advanced healthcare materials, 7(6), 1701024.
Bridge, J. C., Amer, M., Morris, G. E., Martin, N. R. W., Player, D. J., Knox, A. J., . . . & Rose, F. R. (2018). Electrospun gelatin-based scaffolds as a novel 3D platform to study the function of contractile smooth muscle cells in vitro. Biomedical Physics & Engineering Express, Vol 4, No. 4
Gu, L., & Mooney, D. J. (2016). Biomaterials and emerging anticancer therapeutics: engineering the microenvironment. Nature Reviews Cancer, 16(1), 56.
Knight, E., & Przyborski, S. (2015). Advances in 3D cell culture technologies enabling tissue-like structures to be created in vitro. Journal of anatomy, 227(6), 746-756.
Herrmann, D., Conway, J. R., Vennin, C., Magenau, A., Hughes, W. E., Morton, J. P., & Timpson, P. (2014). Three-dimensional cancer models mimic cell-matrix interactions in the tumour microenvironment. Carcinogenesis, 35(8), 1671-1679.
Fong, E. L. S., Lamhamedi-Cherradi, S. E., Burdett, E., Ramamoorthy, V., Lazar, A. J., Kasper, F. K., . . . & Amin, H. M. (2013). Modeling Ewing sarcoma tumors in vitro with 3D scaffolds. Proceedings of the National Academy of Sciences, 201221403.
Nisbet, D. R., Forsythe, J. S., Shen, W., Finkelstein, D. I., & Horne, M. K. (2009). A review of the cellular response on electrospun nanofibers for tissue engineering. Journal of biomaterials applications, 24(1), 7-29.
Pham, Q. P., Sharma, U., & Mikos, A. G. (2006). Electrospinning of polymeric nanofibers for tissue engineering applications: a review. Tissue engineering, 12(5), 1197-1211.
Nisol, B., et al. (2016). Energetics of reactions in a dielectric barrier discharge with argon carrier gas: IV ethyl lactate, Plasma Processes and Polymers, 13(10), 965-969.

1. A method for preparing a three-dimensional (3D) cell culture system, the method comprising:
(i) submitting a solid porous polymeric support comprising electrospun non-woven nanofibers and/or microfibers to cold plasma treatment to obtain a functionalized solid porous polymeric support;
(ii) seeding a first type of cells on the functionalized solid porous polymeric support to attach the first cell type on the solid porous polymeric support; and
(iii) overlaying a biocompatible hydrogel comprising a second type of cells on the solid porous polymeric support of step (ii), wherein at least a portion of the second type of cells migrate into the pores of the solid porous polymeric support, thereby obtaining the 3D culture system.

2. The method of claim 1, wherein the solid porous polymeric support comprises a biocompatible polymer.

3. The method of claim 2, wherein the biocompatible polymer comprises a poly(lactic acid) (PLA), a poly(lactic-co-glycolic acid) (PLGA), a poly(ε-caprolactone) (PCL), a poly(ethylene terephthalate) (PET), a polyethylene glycol (PEG), a polyurethane (PU), or any combinations thereof.

4. The method of claim 3, wherein the biocompatible polymer comprises a PLA, a PCL, a PU, or any combinations thereof.

5. The method of claim 4, wherein the PLA comprises poly-L-Lactide (PLLA).

6. The method of claim 1, wherein the non-woven nanofibers and/or microfibers have an average length ranging from 10 to 5000 μm.

7. The method of claim 1, wherein the non-woven nanofibers and/or microfibers have an average diameter ranging from 50 nm to 5 μm.

8. The method of claim 1, wherein the biocompatible hydrogel comprises collagen, fibrin, fibronectin, hyaluronic acid, gelatin, alginate, a gelatinous protein mixture secreted by Engelbreth-Holm-Swarm (EHS) mouse sarcoma cells, de-cellularized patient extracellular matrix, PEG, hydroxyapatite, chitosan, or any combination thereof.

9. The method of claim 8, wherein the biocompatible hydrogel comprises gelatin, alginate or a mixture thereof.

10. The method of claim 9, wherein the biocompatible hydrogel comprises a mixture of gelatin and alginate.

11. The method of claim 1, wherein the first type of cells comprises epithelial cells, endothelial cells, osteoblasts, stromal cells, or any combination thereof

12. The method of claim 11, wherein the stromal cells are fibroblasts.

13. The method of claim 1, wherein the second type of cells comprises tumor cells, and optionally, tumor stem-like cells, tumor-associated cells, endothelial cells, immune cells, endothelial cells, fibroblasts, epithelial cells, stem cells, or any combination thereof.

14. The method of claim 1, wherein the cold plasma treatment is performed by plasma-enhanced chemical vapor deposition (PECVD).

15. The method of claim 1, wherein the plasma is an O₂ plasma, an NH₃ plasma, or an oxygen-, sulfur- or nitrogen-rich plasma-polymer.

16. The method of claim 15, wherein the oxygen- or nitrogen-rich plasma-polymer is PP-[oxygen-rich ethylene] (PPE:O) or PP-[nitrogen-rich ethylene] (PPE:N).

17. The method of claim 15, wherein the oxygen- or nitrogen-rich plasma polymer is produced using (a) a hydrocarbon source gas comprising butadiene, acetylene, propylene, or butylene; or (b) a volatile organic source gas or vapor that contains a desired oxygen- or nitrogen functionality or functionalities.

18. The method of claim 17, wherein the volatile organic source gas or vapor comprises an organic acid, an alcohol, an ester or an amino-compound.

19. The method of claim 18, wherein the organic acid is acrylic acid, the ester is ethyl lactate (EL), or the amino-compound is allylamine (AAm).

20. The method of claim 1, further comprising culturing the 3D culture system under conditions suitable for cell growth.
